(19) United States
(12) Patent Application Publication
(10) Pub. No.: US 2022/0002690 A1
(43) Pub. Date: Jan. 6, 2022

(54) POLYPEPTIDES HAVING LIPASE ACTIVITY AND USE THEREOF FOR WHEAT SEPARATION

(71) Applicant: NOVOZYMES A/S, Bagsvaerd (DK)

(72) Inventors: James Lavigne, Wake Forest, NC (US); Tucker Shepperd, Raleigh, NC (US); Angela Shows, Raleigh, NC (US); Thomas Patrick Gibbons, Wake Forest, NC (US); Wei Peng, Beijing (CN); Ming Li, Beijing (CN); Tianqi Sun, Aurora (CA); Lars Lehmann Hylling Christensen, Alleroed (DK)

(73) Assignee: NOVOZYMES A/S, Bagsvaerd (DK)

(21) Appl. No.: 17/291,400

(22) PCT Filed: Nov. 20, 2019
(86) PCT No.: PCT/CN2019/119680

§ 371 Date: May 5, 2021

(51) Int. Cl. C12N 9/20; C12N 9/24; C08B 30/04; A23J 1/12; A21D 8/04

(52) U.S. Cl. CPC C12N 9/20; C12N 9/248; A21D 8/042; A23J 1/125; C08B 30/04

(57) ABSTRACT

Provided are improved methods for treating wheat flour with lipase. Further provided are methods for separating wheat flour to provide a gluten fraction, a starch fraction and a fibre fraction where the wheat flour is treated with a selected lipase. Polypeptides having lipase activity, nucleic acid constructs, vectors, and host cells comprising the polynucleotides as well as methods of producing and using the polypeptides are also provided.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to a method for the use of lipases in wheat gluten starch separation. In particular the invention relates to the use of new lipases or the use of lipases not previously used in wheat gluten starch separation, which lipases have particular good performance in such application.

The present invention relates to new polypeptides having lipase activity, and polynucleotides encoding the polypeptides. The invention further relates to nucleic acid constructs, vectors, and host cells comprising the polynucleotides as well as methods of producing and using the polypeptides.

### Description of the Related Art

Before starch, which is an important constituent in the kernels of most crops, such as corn, wheat, rice, sorghum bean, barley or fruit hulls, can be used for conversion of starch into saccharides, such as dextrose, fructose; alcohols, such as ethanol; and sweeteners, the starch must be made available and treated in a manner to provide a high purity starch. If starch contains more than 0.5% impurities, including the proteins, it is not suitable as starting material for starch conversion processes. To provide such pure and high quality starch product starting out from the kernels of crops, the kernels are often milled, as will be described further below. Separation of wheat flour into two or more fractions including a gluten fraction and a starch fraction is a well, known industrial process and in general it is performed using a process containing the steps of
a) Mixing water and wheat flour;
b) Incubating the mixture for a predefined period of time to allow gluten to form a gluten network;
c) Separating the mixture into at least two fractions, a gluten rich fraction and a starch rich fraction; and
d) Optional further purifications of the fractions.

Some enzymes, such as lipases, improve the separation of wheat flour into gluten and starch.

Melis et al. (2017) J. Agric. Food Chem., 65: 1932-1940 describes the use of lipases in the separation of wheat flour into gluten and starch. In the study the authors uses commercial lipases and describes the impact of these commercial lipases on wheat separation. However, the commercial lipases were originally developed for other applications than wheat gluten starch separation, so there is a need for new lipases particular selected for this application.

### SUMMARY OF THE INVENTION

In a first aspect, the invention relates to a process for separating wheat flour into two or more fractions including a gluten fraction and a starch fraction, comprising the steps of:
a) mixing wheat flour and water;
b) adding one or more polypeptide(s) having lipase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction;
   wherein the one or more polypeptide(s) having lipase activity is (are) selected among polypeptides having lipase activity and having a sequence identity to one of SEQ ID NO: 2, 4, 6, 8, 10, 12, 14, 16, 18, 20 or 24 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In a second aspect, the invention relates to a polypeptide having lipase activity, selected from the group consisting of:

(i)
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2;
(b) a polypeptide encoded by a polynucleotide that hybridizes under low stringency conditions with the mature polypeptide coding sequence of SEQ ID NO: 1, or the full-length complement thereof;
(c) a polypeptide encoded by a polynucleotide having 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1;
(d) a variant of the mature polypeptide of SEQ ID NO: 2 comprising a substitution, deletion, and/or insertion at one or more positions; and
(e) a fragment of the polypeptide of (a), (b), (c), or (d) that has lipase activity;

(ii)
(a) a polypeptide having at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4;
(b) a polypeptide encoded by a polynucleotide that hybridizes under medium stringency conditions with the mature polypeptide coding sequence of SEQ ID NO: 3, or the full-length complement thereof;
(c) a polypeptide encoded by a polynucleotide at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 3;
(d) a variant of the mature polypeptide of SEQ ID NO: 4 comprising a substitution, deletion, and/or insertion at one or more positions; and
(e) a fragment of the polypeptide of (a), (b), (c), or (d) that has lipase activity;

(iii)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6;
(b) a polypeptide encoded by a polynucleotide that hybridizes under low stringency conditions with the mature polypeptide coding sequence of SEQ ID NO: 5, the cDNA sequence therefor the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 5 or the cDNA sequence thereof;
(d) a variant of the mature polypeptide of SEQ ID NO: 6 comprising a substitution, deletion, and/or insertion at one or more positions; and
(e) a fragment of the polypeptide of (a), (b), (c), or (d) that has lipase activity;

(iv)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10;
(b) a polypeptide encoded by a polynucleotide that hybridizes under low stringency conditions with the mature polypeptide coding sequence of SEQ ID NO: 9, the cDNA sequence therefor the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 9 or the cDNA sequence thereof;
(d) a variant of the mature polypeptide of SEQ ID NO: 10 comprising a substitution, deletion, and/or insertion at one or more positions; and
(e) a fragment of the polypeptide of (a), (b), (c), or (d) that has lipase activity; and

(v)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 14;
(b) a polypeptide encoded by a polynucleotide that hybridizes under low stringency conditions with the mature polypeptide coding sequence of SEQ ID NO: 13, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 13 or the cDNA sequence thereof;
(d) a variant of the mature polypeptide of SEQ ID NO: 14 comprising a substitution, deletion, and/or insertion at one or more positions; and
(e) a fragment of the polypeptide of (a), (b), (c), or (d) that has lipase activity;

(vi)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 24;
(b) a polypeptide encoded by a polynucleotide that hybridizes under low stringency conditions with the mature polypeptide coding sequence of SEQ ID NO: 23, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 23 or the cDNA sequence thereof;
(d) a variant of the mature polypeptide of SEQ ID NO: 24 comprising a substitution, deletion, and/or insertion at one or more positions; and
(e) a fragment of the polypeptide of (a), (b), (c), or (d) that has lipase activity.

In a third aspect, the invention relates to a composition, e.g. a whole broth formulation or cell culture composition; comprising the polypeptide of the invention.

In a fourth aspect, the invention relates to a polynucleotide encoding the polypeptide of the invention, a nucleic acid construct or expression vector comprising the polynucleotides of the invention, a recombinanthost cell comprising the polynucleotide of the invention and a method of producing a polypeptide having lipase activity using the recombinant host cell of the invention.

### Definitions

Lipase: The term “lipase” means a lipase activity that catalyzes the release of free fatty acids (FFA) from lipids, in particular from wheat lipids. For purposes of the present invention, lipase activity is determined by incubating an enzyme with a 10% wheat slurry for 20 minutes at 38° C., whereafter the amount of released FFA is determined, according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the Lipase activity of the mature polypeptide of SEQ ID NO: 2, SEQ ID NO: 4, SEQ ID NO: 6, SEQ ID NO: 8, SEQ ID NO:10, SEQ ID NO: 12, SEQ ID NO: 14, SEQ ID NO: 16, SEQ ID NO: 18, SEQ ID NO: 20, or SEQ ID NO: 24.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has lipase activity. In one aspect, a fragment consists of a shorter version of the mature polypeptide, e.g., by making N- and/or C-terminal truncations. In one embodiment a fragment comprises amino acids 29 to 301 of SEQ ID NO: 4, or 16 to 288 of SEQ ID NO: 4, or 12 to 285 of SEQ ID NO: 4.

In one embodiment a fragment comprises amino acids 32 to 302 of SEQ ID NO: 6; or 35 to 302 of SEQ ID NO: 6.

In one embodiment a fragment comprises amino acids 88 to 377 of SEQ ID NO: 8.

In one embodiment a fragment comprises amino acids 32 to 343 of SEQ ID NO: 10; or 35 to 306 of SEQ ID NO: 10; or 32 to 306 of SEQ ID NO: 10.

In one embodiment a fragment comprises amino acids105 to 395 of SEQ ID NO: 12.

In one embodiment a fragment comprises amino acids 30 to 150 of SEQ ID NO: 14.

In one embodiment a fragment comprises amino acids 30 to 247 of SEQ ID NO: 16.

In one embodiment a fragment comprises amino acids 34 to 255 of SEQ ID NO: 18.

In one embodiment a fragment comprises amino acids 36 to 288 of SEQ ID NO: 20.

In one embodiment a fragment comprises amino acids 33 to 339 of SEQ ID NO: 24, or 33 to 310 of SEQ ID NO: 24.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 20 to 413 of SEQ ID NO: 2; amino acids 16 to 339 of SEQ ID NO: 4, or 29 to 301 of SEQ ID NO: 4,or 16 to 288 of SEQ ID NO: 4, or 12 to 285 of SEQ ID NO: 4;amino acids 16 to 339 of SEQ ID NO: 6, or 32 to 302 of SEQ ID NO: 6,or 35 to 302 of SEQ ID NO: 6; amino acids 28 to 377 of SEQ ID NO: 8, or 88 to 377 of SEQ ID NO: 8; amino acids 18 to 343 of SEQ ID NO: 10,or 32 to 343 of SEQ ID NO: 10; or 35 to 306 of SEQ ID NO: 10; or 32 to 306 of SEQ ID NO: 10; amino acids 29 to 395 of SEQ ID NO: 12 or 105 to 395 of SEQ ID NO: 12; amino acids 17 to 185 of SEQ ID NO: 14, or 30 to 150 of SEQ ID NO: 14;

amino acids 18 to 247 of SEQ ID NO: 16, or 30 to 247 of SEQ ID NO: 16;amino acids 20 to 255 of SEQ ID NO: 18, or 34 to 255 of SEQ ID NO: 18;amino acids 24 to 228 of SEQ ID NO: 20, or 36 to 288 of SEQ ID NO: 20; amino acids 17 to 339 of SEQ ID NO: 24, or 33 to 339 of SEQ ID NO: 24, or 33 to 310 of SEQ ID NO: 24. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having lipase activity. In one aspect, the mature polypeptide coding sequence is nucleotides 158 to 1342 of SEQ ID NO: 1 and nucleotides 101 to 157 of SEQ ID NO: 1 encode a signal peptide; the mature polypeptide coding sequence is nucleotides 46to 1020 of SEQ ID NO: 3 and nucleotides 1 to 45 of SEQ ID NO: 3 encode a signal peptide; the mature polypeptide coding sequence is nucleotides 546 to 1163 of SEQ ID NO: 5 or the cDNA sequence thereof and nucleotides 501 to 545 of SEQ ID NO: 5 encode a signal peptide; the mature polypeptide coding sequence is nucleotides 82 to 1134 of SEQ ID NO: 7 and nucleotides 1 to 81 of SEQ ID NO: 7 encode a signal peptide; the mature polypeptide coding sequence is nucleotides 152 to 1237 of SEQ ID NO: 9 or the cDNA sequence thereof and nucleotides 101 to 151 of SEQ ID NO: 9 encode a signal peptide; the mature polypeptide coding sequence is nucleotides 185 to 1652 of SEQ ID NO: 11 or the cDNA sequence thereof and nucleotides 101 to 184 of SEQ ID NO: 11 encode a signal peptide; the mature polypeptide coding sequence is nucleotides 90 to 658 of SEQ ID NO: 13 or the cDNA sequence thereof and nucleotides 42 to 89 of SEQ ID NO: 13 encode a signal peptide; the mature polypeptide coding sequence is nucleotides 99 to 866 of SEQ ID NO: 15 or the cDNA sequence thereof and nucleotides 48 to 98 of SEQ ID NO: 15 encode a signal peptide; the mature polypeptide coding sequence is nucleotides 158 to 1044 of SEQ ID NO: 17 or the cDNA sequence thereof and nucleotides 101 to 157 of SEQ ID NO: 17 encode a signal peptide; the mature polypeptide coding sequence is nucleotides 89 to 706 of SEQ ID NO: 19 and nucleotides 20 to 88 of SEQ ID NO: 19 encode a signal peptide; the mature polypeptide coding sequence is nucleotides 49 to 97, and 155 to 1077 of SEQ ID NO: 23, and nucleotides 1 to 48 of SEQ ID NO: 23 encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.]

Subsequence: The term “subsequence” means a polynucleotide having one or more (e.g., several) nucleotides absent from the 5′ and/or 3′ end of a mature polypeptide coding sequence; wherein the subsequence encodes a fragment having lipase activity. In one aspect, a subsequence of SEQ ID NO: 1 contains at least 481 nucleotides (e.g., nucleotides 464 to 946 of SEQ ID NO: 1), at least 603 nucleotides (e.g., nucleotides 398 to 1000 of SEQ ID NO: 1), or at least 1053 nucleotides (e.g., nucleotides 248 to 1300 of SEQ ID NO: 1), a subsequence of SEQ ID NO: 3 contains at least 396 nucleotides (e.g., nucleotides 307 to 702 of SEQ ID NO: 3), at least 603 nucleotides (e.g., nucleotides 223 to 825 of SEQ ID NO: 3), or at least 902 nucleotides (e.g., nucleotides 88 to 990 of SEQ ID NO: 3), a subsequence of SEQ ID NO: 5 contains at least 396 nucleotides (e.g., nucleotides 927 to 1322 of SEQ ID NO: 5), at least 660 nucleotides (e.g., nucleotides 776 to 1436 of SEQ ID NO: 5), or at least 811 nucleotides (e.g., nucleotides 701 to 1511 of SEQ ID NO: 5), a subsequence of SEQ ID NO: 9 contains at least 391 nucleotides (e.g., nucleotides 527 to 917 of SEQ ID NO: 9), at least 566 nucleotides (e.g., nucleotides 389 to 955 of SEQ ID NO: 9), or at least 807 nucleotides (e.g., nucleotides 299 to 1105 of SEQ ID NO: 9), a subsequence of SEQ ID NO: 13 contains at least 383 nucleotides (e.g., nucleotides 147 to 529 of SEQ ID NO: 13), at least 512 nucleotides (e.g., nucleotides 99 to 610 of SEQ ID NO: 13), or at least 642 nucleotides (e.g., nucleotides 99 to 640 of SEQ ID NO: 13).

Variant: The term “variant” means a polypeptide having lipase activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g. several) amino acids, e.g. 1-5 amino acids, adjacent to and immediately following the amino acid occupying a position.

### DETAILED DESCRIPTION OF THE INVENTION

### Wheat Gluten Starch Separation

The invention relates to a method for separating wheat flour into two or more fractions including a gluten fraction and a starch fraction, comprising the steps of:
a) mixing wheatflour and water;
b) adding one or more polypeptide(s) having lipase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction;
wherein the one or more polypeptide(s) having lipase activity is (are) selected among polypeptides having lipase activity and having a sequence identity to one of SEQ ID NO: 2, 4, 6, 8, 10, 12, 14, 16, 18, 20,or 24 of at least 60%.

The wheat flour may in principle be any wheat flour and the invention is not limited to any particular wheat variety, brand or milling procedure as known in the art.

Mixing wheatflour and water is the first step in the method of the invention and has the purpose of enable wheat flour hydration and gluten agglomeration through efficient mixing. This step is well known in the art and is sometimes also called Dough preparation. The step is performed by mixing water and wheatflour under agitation, forming a mixture or dough.

The amount of water added to the wheatflour depends on factors such as the particular process conditions, the particular wheat and the wheat variety used and will readily be determined by the person skilled in the art. Typically the amount of water added is in the range of 0.1-3 Liter per kg wheatflour, preferably 0.5-2.5 Liter per kg wheat flour, preferably 1-2 Liter per kg wheat flour.

The condition such as pH and temperature is typically determined by the ingredients, meaning that the mixing is typically done without any adjustment of pH and temperature, so the pH and temperature is determined by the used raw materials.

According to the invention one or more polypeptides having lipase activity is added to the mixture. The one or more polypeptides having lipase activity may be added together with the wheatflour or it may be added after the wheatflour and water has been mixed. When the one or more polypeptides having lipase activity has been added mixing should continue at least for a sufficient period to secure even distribution in the mixture or dough. The one or more polypeptides having lipase activity is typically added in amounts in the rage of 0.1-500 μg enzyme protein per gram wheat flour (μg EP/g wheat), e.g. in the range of 1-200 μg EP/g wheat, e.g. in the range of 5-100 μg EP/g wheat.

In some embodiments one or more additional enzymes are added together with the one or more polypeptides having lipase activity. In this connection “added together” is intended to mean that the one or more additional enzymes are added simultaneously or sequentially with the one or more polypeptide having lipase activity so that both the one or more additional enzymes and the one or more polypeptides having lipase activity are mixed and evenly distributed in the mixture or dough when the mixing process is completed. Thus, the one and more polypeptides having lipase activity and the one or more additional enzymes may be added as a single composition or as two or more separate compositions each comprising one or more enzymes.

The one or more additional enzymes may be selected among cellulases, xylanases, proteases amylases, arabinofuranosidases.

In a preferred embodiment a polypeptide having xylanase activity is added together with the polypeptide having lipase activity. The polypeptide having xylanase activity may be selected among GH8, GH10 or GH11 xylanases.

A preferred xylanase according to the invention is the GH10 xylanase disclosed in WO 97/021785.

Another preferred polypeptide having xylanase activity is the GH8 xylanase disclosed in WO 2011/070101. Preferably the polypeptide having GH8 xylanase activity is present in an amount of preferably 0.0005 to 1.5 mg enzyme protein per g wheat flour, preferably 0.001 to 1 mg enzyme protein per g wheat flour, preferably 0.01 to 0.5 mg enzyme protein per g wheat flour, preferably 0.025 to 0.25 mg enzyme protein per g wheat flour.

In one preferred embodiment the polypeptide having xylanase activity is a the xylanase is a GH8 xylanase and have a sequence identity to SEQ ID NO: 21 of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another preferred embodiment the polypeptide having xylanase activity is a the xylanase is a GH10 xylanase and have a sequence identity to SEQ ID NO: 22 of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

Incubating the mixture for a predefined period of time. When the mixing is complete the mixture or dough is incubated in a predefined period to allow the gluten to form gluten network. Further the one or more polypeptides having lipase activity will during this period hydrolyse the lipids in the mixture or dough and the optional additional enzymes may act upon their substrates during this incubation period. This is also called dough maturation and is typically done in a maturation tank. Typically the incubation is done at ambient temperature i.e. without temperature regulation. Thus the incubation typically takes place at a temperature in the range of 5-50° C. , preferably in the range of 15-40° C. and most preferred in the range of 20-35° C.

The incubation is performed for a sufficient time to allow the gluten network to form and the duration is easily determined by the person skilled in the art. The mixture may be performed for a period in the range of 5 minutes to 8 hours, e.g. in the range of 15 minutes to 4 hours.

Separating the mixture into two or more fractions including a gluten rich fraction and a starch rich fraction. After the incubation period the mixture is separated into two or more fractions including a starch rich fraction and a gluten rich fraction.

A starch rich fraction is in this application intended to mean a fraction that comprises at least 50% (w/w) starch, preferably at least 60% (w/w) starch, preferably at least 70% (w/w) starch, preferably at least 80% (w/w) starch, preferably at least 90% (w/w) starch, calculated based on the dry matter of the fraction.

A gluten rich fraction is in this application intended to mean a fraction that comprises at least 50% (w/w) gluten, preferably at least 60% (w/w) gluten, preferably at least 70% (w/w) gluten, preferably at least 80% (w/w) gluten, preferably at least 90% (w/w) gluten, calculated based on the dry matter of the fraction.

The separation step may be performed based on differences in solubility and density using methods and equipment known in the art.

In a preferred embodiment the separation step is performed using a 3 phase separator process separating the mixture or dough into a starch rich fraction; a gluten rich fraction; and a pentosan fraction having a high content of fibers, in particular pentosans such as arabinoxylans.

After the separation step separating the mixture/dough into two or more fractions including a gluten rich fraction and a starch rich fraction, each of these fractions may be subjected to additional separation steps in order to purify the fractions even further and avoid loss. Such operations are known in the art and are e.g. known as gluten washing, starch washing and fiber washing and are typically performed using a number of decanters, sedicanters, centrifuges, screens, hydrocyclones etc. as known in the art.

The separation steps have been completed and the two or more fractions have obtained their intended purity the fraction is recovered, typically by removing excess water and obtaining the fractions in dry stable form. Alternatively, the obtained fractions may immediately be further processed without drying.

In a further aspect the invention relates to the use of one or more polypeptides having lipase activity is (are) selected among polypeptides having lipase activity and having a sequence identity to one of SEQ ID NO:2, 4, 6, 8, 10, 12, 14, 16, 18, 20, or 24 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% in a process for separating wheat into a gluten fraction, a starch fraction and a fibre fraction, preferably the use of one or more polypeptides having lipase activity is (are) selected among polypeptides having lipase activity and having a sequence identity to one of SEQ ID NO:2, 4, 6, 8, 10, 12, 14, 16, 18, 20,or 24 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% in combination with one or more polypeptides having xylanase activity.

There are several technical benefits to be derived from the process of the invention, including, an improved separation; preferably the process provides a reduced viscosity in the wheatflour slurry as determined herein and/or a higher protein recovery as determined herein. This has been reflected in that the capacity in the first separation step separating the mixture or dough into two or more fractions including a starch rich fraction and a gluten rich fraction compared with same. For example, using a 3-phase separator is was shown that the capacity increased with more than 20% using a lipase according to the invention compared with corresponding separation done without addition of lipase according to the invention.

### Polypeptides Having Lipase Activity

In an embodiment, the present invention relates to polypeptides having a sequence identity to the mature polypeptide of SEQ ID NO: 2 of at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to10 amino acids, e.g., 1, 2, 3, 4, 5, 6, 7, 8, 9, or 10, from the mature polypeptide of SEQ ID NO: 2.

In a particular embodiment, the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the lipaseactivity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the activity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 2.

In a particular embodiment the invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 2.

In an embodiment, the polypeptide has been isolated. A polypeptide of the present invention preferably comprises or consists of the amino acid sequence of SEQ ID NO: 2 or an allelic variant thereof; or is a fragment thereof having lipase activity. In another aspect, the polypeptide comprises or consists of the mature polypeptide of SEQ ID NO: 2. In another aspect, the polypeptide comprises or consists of amino acids 20 to 413 of SEQ ID NO: 2.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, or (ii) the full-length complement of (i) (Sambrook et al., 1989, Molecular Cloning, A Laboratory Manual, 2d edition, Cold Spring Harbor, New York). In an embodiment, the polypeptide has been isolated.

The polynucleotide of SEQ ID NO: 1 or a subsequence thereof, as well as the polypeptide of SEQ ID NO: 2 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 101 to 1347, nucleotides 158 to 1347, nucleotides 300 to 1200, or nucleotides 500 to 1000 of SEQ ID NO: 1. In another aspect, the nucleic acid probe is a polynucleotide that encodes the polypeptide of SEQ ID NO: 2; the mature polypeptide thereof; or a fragment thereof. In another aspect, the nucleic acid probe is SEQ ID NO: 1.

In another embodiment, the present invention relates to a polypeptide having lipase activity encoded by a polynucleotide having a sequence identity to the mature polypeptide coding sequence of SEQ ID NO: 1 of at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 2 comprising a substitution, deletion, and/or insertion at one or more (e.g., several) positions. In an embodiment, the number of amino acid substitutions, deletions and/or insertions introduced into the mature polypeptide of SEQ ID NO: 2 is up to 10, e.g., 1, 2, 3, 4, 5, 6, 7, 8, 9, or 10.The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In an embodiment, the present invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to 10 amino acids, e.g., 1, 2, 3, 4, 5, 6, 7, 8, 9, or 10, from the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 4.

In a particular embodiment the invention relates to polypeptides having a sequence identity to the mature polypeptide of SEQ ID NO: 4 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 4.

In an embodiment, the polypeptide has been isolated. A polypeptide of the present invention preferably comprises or consists of the amino acid sequence of SEQ ID NO: 4 or an allelic variant thereof; or is a fragment thereof having lipase activity. In another aspect, the polypeptide comprises or consists of the mature polypeptide of SEQ ID NO: 4. In another aspect, the polypeptide comprises or consists of amino acids 16 to 339 of SEQ ID NO: 4, amino acids 29 to 301 of SEQ ID NO: 4,or 16 to 288 of SEQ ID NO: 4, or 12 to 285 of SEQ ID NO: 4.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 3, or (ii) the full-length complement of (i) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 3 or a subsequence thereof, as well as the polypeptide of SEQ ID NO: 4 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 3 or a subsequence thereof, the carrier material is used in a Southern blot.

For purposes of the present invention, hybridization indicates that the polynucleotide hybridizes to a labeled nucleic acid probe corresponding to (i) SEQ ID NO: 3; (ii) the mature polypeptide coding sequence of SEQ ID NO: 3; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 1 to 1020, nucleotides 46 to 1000, nucleotides 200 to 800, or nucleotides 300 to 700 of SEQ ID NO: 3. In another aspect, the nucleic acid probe is a polynucleotide that encodes the polypeptide of SEQ ID NO: 4; the mature polypeptide thereof; or a fragment thereof. In another aspect, the nucleic acid probe is SEQ ID NO: 3.

In another embodiment, the present invention relates to an polypeptide having lipase activity encoded by a polynucleotide having a sequence identity to the mature polypeptide coding sequence of SEQ ID NO: 3 of at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 4 comprising a substitution, deletion, and/or insertion at one or more (e.g., several) positions. In an embodiment, the number of amino acid substitutions, deletions and/or insertions introduced into the mature polypeptide of SEQ ID NO: 4 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In an embodiment, the present invention relates to polypeptides having a sequence identity to the mature polypeptide of SEQ ID NO: 6 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to 10 amino acids, e.g., 1, 2, 3, 4, 5, 6, 7, 8, 9, or 10, from the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the lipase activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the lipaseactivity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 6.

In an embodiment, the polypeptide has been isolated. A polypeptide of the present invention preferably comprises or consists of the amino acid sequence of SEQ ID NO: 6 or an allelic variant thereof; or is a fragment thereof having lipase activity. In another aspect, the polypeptide comprises or consists of the mature polypeptide of SEQ ID NO: 6. In another aspect, the polypeptide comprises or consists of amino acids 16 to 339 of SEQ ID NO: 6, amino acids 32 to 302 of SEQ ID NO: 6; or 35 to 302 of SEQ ID NO: 6.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 5, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 5 or a subsequence thereof, as well as the polypeptide of SEQ ID NO: 6 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 5 or a subsequence thereof, the carrier material is used in a Southern blot.

For purposes of the present invention, hybridization indicates that the polynucleotide hybridizes to a labeled nucleic acid probe corresponding to (i) SEQ ID NO: 5; (ii) the mature polypeptide coding sequence of SEQ ID NO: 5; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 501 to 1631, nucleotides 546 to 1631, nucleotides 648 to 813, or nucleotides 872 to 1631 of SEQ ID NO: 5. In another aspect, the nucleic acid probe is a polynucleotide that encodes the polypeptide of SEQ ID NO: 6; the mature polypeptide thereof; or a fragment thereof. In another aspect, the nucleic acid probe is SEQ ID NO: 6 or the cDNA sequence thereof.

In another embodiment, the present invention relates to a polypeptide having lipase activity encoded by a polynucleotide having a sequence identity to the mature polypeptide coding sequence of SEQ ID NO: 5or the cDNA sequence thereof, of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 6 comprising a substitution, deletion, and/or insertion at one or more (e.g., several) positions. In an embodiment, the number of amino acid substitutions, deletions and/or insertions introduced into the mature polypeptide of SEQ ID NO: 6 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In an embodiment, the present invention relates to polypeptides having a sequence identity to the mature polypeptide of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to 10 amino acids, e.g., 1, 2, 3, 4, 5, 6, 7, 8, 9, or 10, from the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 10.

In a particular embodiment the invention relates to polypeptides having a sequence identity to the mature polypeptide of SEQ ID NO: 10 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 10.

In an embodiment, the polypeptide has been isolated. A polypeptide of the present invention preferably comprises or consists of the amino acid sequence of SEQ ID NO: 10 or an allelic variant thereof; or is a fragment thereof having lipase activity. In another aspect, the polypeptide comprises or consists of the mature polypeptide of SEQ ID NO: 10. In another aspect, the polypeptide comprises or consists of amino acids 18 to 343 of SEQ ID NO: 10, amino acids 32 to 343 of SEQ ID NO: 10; or 35 to 306 of SEQ ID NO: 10; or 32 to 306 of SEQ ID NO: 10.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 9, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 9 or a subsequence thereof, as well as the polypeptide of SEQ ID NO: 10 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 9 or a subsequence thereof, the carrier material is used in a Southern blot.

For purposes of the present invention, hybridization indicates that the polynucleotide hybridizes to a labeled nucleic acid probe corresponding to (i) SEQ ID NO: 9; (ii) the mature polypeptide coding sequence of SEQ ID NO: 9; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 101 to 1237, nucleotides 152 to 1237, nucleotides 246 to 411, or nucleotides 466 to 1237 of SEQ ID NO: 9. In another aspect, the nucleic acid probe is a polynucleotide that encodes the polypeptide of SEQ ID NO: 10; the mature polypeptide thereof; or a fragment thereof. In another aspect, the nucleic acid probe is SEQ ID NO: 9or the cDNA sequence thereof.

In another embodiment, the present invention relates to a polypeptide having lipase activity encoded by a polynucleotide having a sequence identity to the mature polypeptide coding sequence of SEQ ID NO: 9or the cDNA sequence thereof of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 10 comprising a substitution, deletion, and/or insertion at one or more (e.g., several) positions. In an embodiment, the number of amino acid substitutions, deletions and/or insertions introduced into the mature polypeptide of SEQ ID NO: 10 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In an embodiment, the present invention relates to a polypeptide having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to 10 amino acids, e.g., 1, 2, 3, 4, 5, 6, 7, 8, 9, or 10, from the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 70% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 90% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 14.

In a particular embodiment the invention relates to polypeptides having a sequence identity to the mature polypeptide of SEQ ID NO: 14 of at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 14.

In an embodiment, the polypeptide has been isolated. A polypeptide of the present invention preferably comprises or consists of the amino acid sequence of SEQ ID NO: 14 or an allelic variant thereof; or is a fragment thereof having lipase activity. In another aspect, the polypeptide comprises or consists of the mature polypeptide of SEQ ID NO: 14. In another aspect, the polypeptide comprises or consists of amino acids 17 to 185 of SEQ ID NO: 14, or amino acids 30 to 150 of SEQ ID NO: 14.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 13, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 13 or a subsequence thereof, as well as the polypeptide of SEQ ID NO: 14 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 13 or a subsequence thereof, the carrier material is used in a Southern blot.

For purposes of the present invention, hybridization indicates that the polynucleotide hybridizes to a labeled nucleic acid probe corresponding to (i) SEQ ID NO: 13; (ii) the mature polypeptide coding sequence of SEQ ID NO: 13; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 42 to 658, nucleotides 90 to 658, nucleotides 90 to 382, or nucleotides 442 to 658 of SEQ ID NO: 13. In another aspect, the nucleic acid probe is a polynucleotide that encodes the polypeptide of SEQ ID NO: 14; the mature polypeptide thereof; or a fragment thereof. In another aspect, the nucleic acid probe is SEQ ID NO: 13or the cDNA sequence thereof.

In another embodiment, the present invention relates to a polypeptide having lipase activity encoded by a polynucleotide having a sequence identity to the mature polypeptide coding sequence of SEQ ID NO: 13or the cDNA sequence thereof of at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 14 comprising a substitution, deletion, and/or insertion at one or more (e.g., several) positions. In an embodiment, the number of amino acid substitutions, deletions and/or insertions introduced into the mature polypeptide of SEQ ID NO: 14 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In an embodiment, the present invention relates to a polypeptide having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, which have lipase activity. In one aspect, the polypeptides differ by up to 10 amino acids, e.g., 1, 2, 3, 4, 5, 6, 7, 8, 9, or 10, from the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 70% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 75% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 80% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 85% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 90% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 95% of the lipaseactivity of the mature polypeptide of SEQ ID NO: 24.

In a particular embodiment the invention relates to polypeptides having a sequence identity to the mature polypeptide of SEQ ID NO: 24 of at least 90%, at least 91%, at least 92%, at least 93%, at least 94%, at least 95%, at least 96%, at least 97%, at least 98%, at least 99%, or 100%, and wherein the polypeptide has at least at least 100% of the lipaseactivity of the mature polypeptide of SEQ ID NO: 24.

In an embodiment, the polypeptide has been isolated. A polypeptide of the present invention preferably comprises or consists of the amino acid sequence of SEQ ID NO: 24 or an allelic variant thereof; or is a fragment thereof having lipase activity. In another aspect, the polypeptide comprises or consists of the mature polypeptide of SEQ ID NO: 24. In another aspect, the polypeptide comprises or consists of amino acids 17 to 339 of SEQ ID NO: 24, or amino acids 33 to 339 of SEQ ID NO: 24, or amino acids 33 to 310 of SEQ ID NO: 24.

In another embodiment, the present invention relates to a polypeptide having lipase activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 23, (ii) the cDNA sequence thereof, or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 23 or a subsequence thereof, as well as the polypeptide of SEQ ID NO: 24 or a fragment thereof may be used to design nucleic acid probes to identify and clone DNA encoding polypeptides having lipase activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having lipase activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 23 or a subsequence thereof, the carrier material is used in a Southern blot.

For purposes of the present invention, hybridization indicates that the polynucleotide hybridizes to a labeled nucleic acid probe corresponding to (i) SEQ ID NO: 23; (ii) the mature polypeptide coding sequence of SEQ ID NO: 23; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides 49 to 97, and 155 to 1077 of SEQ ID NO: 23. In another aspect, the nucleic acid probe is a polynucleotide that encodes the polypeptide of SEQ ID NO: 24; the mature polypeptide thereof; or a fragment thereof. In another aspect, the nucleic acid probe is SEQ ID NO: 23or the cDNA sequence thereof.

In another embodiment, the present invention relates to a polypeptide having lipase activity encoded by a polynucleotide having a sequence identity to the mature polypeptide coding sequence of SEQ ID NO: 23or the cDNA sequence thereof of at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 24 comprising a substitution, deletion, and/or insertion at one or more (e.g., several) positions. In an embodiment, the number of amino acid substitutions, deletions and/or insertions introduced into the mature polypeptide of SEQ ID NO: 24 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Neret al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol.3: 568-576; Svetinaet al., 2000, J. Biotechnol.76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racieet al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Lipase Activity

A polypeptide having lipase activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In one aspect the polypeptides having lipase activity of the present invention is derived from a strain belonging to the Plectosphaerella, Nectria, Acremonium, Mucor, Fusarium, Trichoderma, Penicillium or Humicola genera.

In another aspect, the polypeptide is a Plectosphaerellaalismatis polypeptide, a Mucor wutungkiao polypeptide, a Mucor circinnelloides polypeptide, a Fusarium solani polypeptide, a Trichoderma atroviride polypeptideor a Humicolain solens polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), CentraalbureauVoorSchimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences. In one embodiment the one or more control sequences may be foreign (heterologous) to the polynucleotide of the invention.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylBgenes, Bacillus thuringiensis crylllA gene (Agaisse and Lereclus, 1994, Molecular Microbiology 13: 97-107), E. coli lac operon, E. coli trc promoter (Egon et al., 1988, Gene 69: 301-315), Streptomyces coelicoloragarase gene (dagA), and prokaryotic beta-lactamase gene (Villa-Kamaroffet al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulansacetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO96/00787), Fusarium venenatum amyloglucosidase (WO00/56900), Fusarium venenatum Daria (WO00/56900), Fusarium venenatum Quinn (WO00/56900), Rhizomucormiehei lipase, Rhizomucormiehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanoset al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulansacetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanoset al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis crylllA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicolainsolens cellulase, Humicolainsolens endoglucanase V, Humicolalanuginosa lipase, and Rhizomucormiehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanoset al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthorathermophila laccase (WO95/33836), Rhizomucormiehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. In one embodiment the one or more control sequences may be foreign (heterologous) to the polynucleotide of the invention. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS(acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrGgenes.

The selectable marker may be a dual selectable marker system as described in WO 2010/039889. In one aspect, the dual selectable marker is anhph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAN/1111 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. In one embodiment the polynucleotide of the present invention may be foreign (heterologous) to the host cell. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodieret al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti(Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowialipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkanderaadusta, Ceriporiopsisaneirina, Ceriporiopsiscaregiea, Ceriporiopsisgilvescens, Ceriporiopsispannocinta, Ceriporiopsisrivulosa, Ceriporiopsissubrufa, Ceriporiopsissubvermispora, Chrysosporiuminops, Chrysosporiumkeratinophilum, Chrysosporiumlucknowense, Chrysosporiummerdarium, Chrysosporiumpannicola, Chrysosporiumqueenslandicum, Chrysosporiumtropicum, Chrysosporiumzonatum, Coprinus cinereus, Coriolushirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicolainsolens, Humicolalanuginosa, Mucor miehei, Myceliophthorathermophila, Neurospora crassa, Penicillium purpurogenum, Phanerochaetechrysosporium, Phlebia radiata, Pleurotusetyngii, Thielaviaterrestris, Trametesvillosa, Trametes versicolor, Trichoderma harzianum, Trichoderma Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP238023, Yeltonet al., 1984, Proc. Natl. Acad. Sci. USA 81: 1470-1474, and Christensen et al., 1988, Bio/Technology 6: 1419-1422. Suitable methods for transforming Fusarium species are described by Malardieret al., 1989, Gene 78: 147-156, and WO96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnenet al., 1978, Proc. Natl. Acad. Sci. USA 75: 1920.

### Methods of Production

The present invention also relates to methods of producing a polypeptide of the present invention, comprising(a) cultivating a cell, which in its wild-type form produces the polypeptide, under conditions conducive for production of the polypeptide; and optionally, (b) recovering the polypeptide.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The present invention also relates to compositions comprising a polypeptide of the present invention. Preferably, the compositions are enriched in such a polypeptide. The term “enriched” indicates that the lipase activity of the composition has been increased, e.g., with an enrichment factor of at least 1.1.

The compositions may comprise a polypeptide of the present invention as the major enzymatic component, e.g., a mono-component composition. Alternatively, the compositions may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

The invention is further described in the following numbered embodiments.
Embodiment 1. A process for separating wheat flour into two or more fractions including a gluten fraction and a starch fraction, comprising the steps of:
(a) mixing wheat flour and water;
(b) adding one or more polypeptide(s) having lipase activity;
(c) incubating the mixture for a predefined period of time;
(d) separating the mixture into two or more fractions including a gluten rich fraction and a starch rich fraction; and
(e) recovering the two or more fractions including a gluten rich fraction and a starch rich fraction;
   wherein the one or more polypeptide(s) having lipase activity is (are) selected among polypeptides having lipase activity and having a sequence identity to one of SEQ ID NO: 2, 4, 6, 8, 10, 12, 14, 16, 18, 20 or 24 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.
Embodiment 2. The process of embodiment 1, where in step a) the water and wheat flour is mixed in a ratio of 0.1-3 Liter of water per kg wheat flour, preferably 0.5-2.5 Liter of water per kg wheat flour, preferably 1-2 Liter of water per kg wheat flour.
Embodiment 3. The process of embodiment 1 or 2, wherein the one or more polypeptides having lipase activity is added in amounts of 0.1-500 μg enzyme protein per gram wheat flour (μg EP/g wheat), e.g. in the range of 1-200 μg EP/g wheat flour, e.g. in the range of 5-100 μg EP/g wheat flour.
Embodiment 4. The process according to any of the preceding embodiments, wherein a xylanase is added together with the one or more polypeptides having lipase activity.
Embodiment 5. The process of embodiment 4, wherein the xylanase is selected from xylanases belonging to the GH8, GH10 or GH11 families.
Embodiment 6. The process of embodiment 5, wherein the xylanase is a GH8 xylanase and have a sequence identity to SEQ ID NO: 21 of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.
Embodiment 7. The process of embodiment 5, wherein the xylanase is a GH10 xylanase and have a sequence identity to SEQ ID NO: 22 of at least 60% e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.
Embodiment 8. The process according to any one of embodiment 4-7, wherein the xylanase is added in an amount of 0.0005 to 1.5 mg enzyme protein per g wheat flour, preferably 0.001 to 1 mg enzyme protein per g wheat flour, preferably 0.01 to 0.5 mg enzyme protein per g wheat flour, preferably 0.025 to 0.25 mg enzyme protein per g wheat flour.
Embodiment 9. The process according to any of the preceding embodiments, wherein the incubation in step c) is performed for 5 minutes to 8 Hours, preferably 15 minutes to 4 Hours.
Embodiment 10. The process according to any of the preceding embodiments, wherein step d) is performed in a three-phase separator and provides a gluten rich fraction, a starch rich fraction and a pentosane/fiber rich fraction.
Embodiment 11. The process according to any of the preceding embodiments, having one or more benefits compared to a similar process without addition of a polypeptide having lipase activity selected among: reduced viscosity in the wheat flour slurry, higher protein recovery and higher throughput in the separation step.
Embodiment 12. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2; and
(b) a fragment of the polypeptide of (a) that has lipase activity.
Embodiment 13. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4; and
(b) a fragment of the polypeptide of (a) that has lipase activity.
Embodiment 14. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6; and
(b) a fragment of the polypeptide of (a) that has lipase activity.
Embodiment 15. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 8; and
(b) a fragment of the polypeptide of (a) that has lipase activity.
Embodiment 16. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10; and
(b) a fragment of the polypeptide of (a) that has lipase activity.
Embodiment 17. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 12; and
(b) a fragment of the polypeptide of (a) that has lipase activity.
Embodiment 18. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 14; and
(b) a fragment of the polypeptide of (a) that has lipase activity.
Embodiment 19. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 16; and
(b) a fragment of the polypeptide of (a) that has lipase activity.
Embodiment 20. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 18; and
(b) a fragment of the polypeptide of (a) that has lipase activity.
Embodiment 21. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 20; and
(b) a fragment of the polypeptide of (a) that has lipase activity.
Embodiment 22. The process of embodiment 1, wherein the lipase is selected from:
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 24; and
(b) a fragment of the polypeptide of (a) that has lipase activity.
Embodiment 23. The process according to any of the embodiments 12 to 22, wherein the fragments are selected from the group consisting of:
(a) amino acids 29 to 301 of SEQ ID NO: 4,or 16 to 288 of SEQ ID NO: 4, or 12 to 285 of SEQ ID NO: 4;
(b) amino acids 32 to 302 of SEQ ID NO: 6; or 35 to 302 of SEQ ID NO: 6;
(c) amino acids 88 to 377 of SEQ ID NO: 8;
(d) amino acids 32 to 343 of SEQ ID NO: 10; or 35 to 306 of SEQ ID NO: 10; or 32 to 306 of SEQ ID NO: 10;
(e) amino acids105 to 395 of SEQ ID NO: 12;
(f) amino acids 30 to 150 of SEQ ID NO: 14;
(g) amino acids 30 to 247 of SEQ ID NO: 16;
(h) amino acids 34 to 255 of SEQ ID NO: 18;
(i) amino acids 36 to 288 of SEQ ID NO: 20; and
(j) amino acids 33 to 339 of SEQ ID NO: 24, or 33 to 310 of SEQ ID NO: 24.
Embodiment 24. A polypeptide having lipase activity, selected from the group consisting of:

(i)
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2;
(b) a polypeptide encoded by a polynucleotide that hybridizes under low stringency conditions with the mature polypeptide coding sequence of SEQ ID NO: 1, or the full-length complement thereof;
(c) a polypeptide encoded by a polynucleotide having 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1;
(d) a variant of the mature polypeptide of SEQ ID NO: 2 comprising a substitution, deletion, and/or insertion at one or more positions; and
(e) a fragment of the polypeptide of (a), (b), (c), or (d) that has lipase activity;

(ii)
(a) a polypeptide having at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4;
(b) a polypeptide encoded by a polynucleotide that hybridizes undermedium stringency conditions with the mature polypeptide coding sequence of SEQ ID NO: 3, or the full-length complement thereof;
(c) a polypeptide encoded by a polynucleotide at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 3;
(d) a variant of the mature polypeptide of SEQ ID NO: 4 comprising a substitution, deletion, and/or insertion at one or more positions; and
(e) a fragment of the polypeptide of (a), (b), (c), or (d) that has lipase activity;

(iii)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6;
(b) a polypeptide encoded by a polynucleotide that hybridizes under low stringency conditions with the mature polypeptide coding sequence of SEQ ID NO: 5, the cDNA sequence therefor the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 5 or the cDNA sequence thereof;
(d) a variant of the mature polypeptide of SEQ ID NO: 6 comprising a substitution, deletion, and/or insertion at one or more positions; and
(e) a fragment of the polypeptide of (a), (b), (c), or (d) that has lipase activity;

(iv)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10;
(b) a polypeptide encoded by a polynucleotide that hybridizes under low stringency conditions with the mature polypeptide coding sequence of SEQ ID NO: 9, the cDNA sequence therefor the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 9 or the cDNA sequence thereof;
(d) a variant of the mature polypeptide of SEQ ID NO: 10 comprising a substitution, deletion, and/or insertion at one or more positions; and
(e) a fragment of the polypeptide of (a), (b), (c), or (d) that has lipase activity; and

(v)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 14;
(b) a polypeptide encoded by a polynucleotide that hybridizes under low stringency conditions with the mature polypeptide coding sequence of SEQ ID NO: 13, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 13 or the cDNA sequence thereof;
(d) a variant of the mature polypeptide of SEQ ID NO: 14 comprising a substitution, deletion, and/or insertion at one or more positions; and
(e) a fragment of the polypeptide of (a), (b), (c), or (d) that has lipase activity;

(vi)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 24;
(b) a polypeptide encoded by a polynucleotide that hybridizes under low stringency conditions with the mature polypeptide coding sequence of SEQ ID NO: 23, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 23 or the cDNA sequence thereof;
(d) a variant of the mature polypeptide of SEQ ID NO: 24 comprising a substitution, deletion, and/or insertion at one or more positions; and
(e) a fragment of the polypeptide of (a), (b), (c), or (d) that has lipase activity.
Embodiment 25. The polypeptide of embodiment24, comprising or consisting of SEQ ID NO: 2, 4, 6, 10, 14 or 24; or the mature polypeptide of SEQ ID NO: 2, 4, 6, 10, 14 or 24.
Embodiment 26. The polypeptide of embodiment25, wherein the mature polypeptide is amino acids 20 to 413 of SEQ ID NO: 2; amino acids 16 to 339 of SEQ ID NO: 4; amino acids 16 to 339 of SEQ ID NO: 6; amino acids 18 to 343 of SEQ ID NO: 10, amino acids 17 to 185 of SEQ ID NO: 14; or amino acids 20 to 339 of SEQ ID NO: 24.
Embodiment 27. The polypeptide of embodiment24-26, which is a fragment of SEQ ID NO: 2, 4, 6, 10, 14 or 24, wherein the fragment has lipase activity.
Embodiment 28. The polypeptide of embodiment 27, wherein the fragment is selected from the groups consisting of:
   (i) amino acids 29 to 301 of SEQ ID NO: 4,or 16 to 288 of SEQ ID NO: 4, or 12 to 285 of SEQ ID NO: 4;
   (ii) amino acids 32 to 302 of SEQ ID NO: 6; or 35 to 302 of SEQ ID NO: 6;
   (iii) amino acids 32 to 343 of SEQ ID NO: 10; or 35 to 306 of SEQ ID NO: 10; or 32 to 306 of SEQ ID NO: 10;
   (iv) amino acids 30 to 150 of SEQ ID NO: 14; and
   (v) amino acids 33 to 339 of SEQ ID NO: 24, or 33 to 310 of SEQ ID NO: 24.
Embodiment 29. A composition comprising the polypeptide of any of embodiments 24-28.
Embodiment 30. A whole broth formulation or cell culture composition comprising the polypeptide of any of embodiments 24-28.
Embodiment 31. A polynucleotide encoding the polypeptide of any of embodiments 24-28.
Embodiment 32. A nucleic acid construct or expression vector comprising the polynucleotide of embodiment 31 operably linked to one or more control sequences that direct the production of the polypeptide in an expression host.
Embodiment 33. A recombinant host cell comprising the polynucleotide of embodiment 31 operably linked to one or more control sequences that direct the production of the polypeptide.
Embodiment 34. A method of producing a polypeptide having lipase activity, comprising cultivating the host cell of embodiment 33 under conditions conducive for production of the polypeptide.
Embodiment 35. The method of embodiment 34, further comprising recovering the polypeptide.

The present invention is further described by the following examples that should not be construed as limiting the scope of the invention.

### EXAMPLES

### Enzymes:

Lipase: Lipolase™, available from Novozymes A/S, Bagsværd Denmark

### Strains:

Escherichia coli Top-10 strain purchased from TIANGEN (TIANGEN Biotech Co. Ltd., Beijing, China) was used to propagate our expression vector.
Aspergillus oryzae MT3568 strain was used for heterologous expression of the gene encoding a polypeptide having homology with polypeptides with lipase activity. A. oryzae MT3568 is an amdS (acetamidase) disrupted gene derivative of A. oryzae JaL355 (WO02/40694) in which pyrGauxotrophy was restored by disrupting the A. oryzae acetamidase (amdS) gene with the pyrG gene.

### Media

YPM medium was composed of 10 g yeast extract, 20 g Bacto-peptone, 20 g maotose, and deionised water to 1000 ml.

LB plates were composed of 10 g of Bacto-tryptone, 5 g of yeast extract, 10 g of sodium chloride, 15 g of Bacto-agar, and deionised water to 1000 ml.

LB medium was composed of 1 g of Bacto-tryptone, 5 g of yeast extract, and 10 g of sodium chloride, and deionised water to 1000 ml.

COVE sucrose plates were composed of 342 g of sucrose, 20 g of agar powder, 20 ml of COVE salt solution, and deionized water to 1 liter. The medium was sterilized by autoclaving at 15 psi for 15 minutes. The medium was cooled to 60° C. and 10 mM acetamide, 15 mM CsCl, Triton X-100 (50 μl/500 ml) were added.

COVE-2 plate/tube for isolation: 30 g/L sucrose, 20 ml/L COVE salt solution, 10 mM acetamide, 30 g/L noble agar (Difco, Cat#214220).

COVE salt solution was composed of 26 g of MgSO₄.7H₂O, 26 g of KCL, 26 g of KH₂PO₄, 50 ml of COVE trace metal solution, and deionised water to 1000 ml.

COVE trace metal solution was composed of 0.04 g of Na₂B₄O₇.10H₂O, 0.4 g of CuSO₄.5H₂O, 1.2 g of FeSO₄.7H₂O, 0.7 g of MnSO₄.H₂O, 0.8 g of Na₂MoO₄.2H₂O, 10 g of ZnSO₄.7H₂O, and deionised water to 1000 ml.

Mature polypeptides:

The mature form of the lipase polypeptides and fragments thereof having lipase activity was determined using well know techniques in the art, by e.g., precise intact mass determined by LC-MS after de-glycosylation with EndoH.

### Example 1

### Wheat Protein Recovery

Approximately 250 g of wheat flour was transferred into an appropriately sized mixing bowl. Then 150 mL of heated tap water was added to the flour. A. aculeatus xylanase (disclosed in WO 2005/118769) was dosed at 15 μg EP/g flour and lipase (Lipolase™) was dosed at 3 ug EP/g flour. A control with xylanase but without lipase was included. The contents were mixed for 4 minutes with a stand-mixer (Kitchen Aid Model: Ultra Power 300 watts max) equipped with a dough hook and set to a speed of 4. Afterwards, the formed dough was allowed to rest for 8 minutes, then 250 mL of heated tap water was added to the mixing bowl. The contents were mixed for an additional 25 minutes with a flat beater at a speed setting of stir. Then 1000 mL of heated tap water was added to the mixing bowl. The contents were stirred again for 35 minutes, then poured over a 425-um sieve. The sieve was vibrated to enable separation. Approximately 1000 mL of heated tap water was added to the mixing bowl for a final rinse, then poured over said sieve and vibrated as before. The material remaining on top of the sieve was recovered and then analyzed for protein content using a total nitrogen analyzer (LECO corporation model FP628). The results are shown in Table 1, where itis clear that the lipase improved the protein recovery about 1.5-2%.

TABLE 1

| Treatment | % Protein recovery |
|---|---|
| Xylanase | 17 |
| Xylanase + Lipolase ™ | 18.8 |

### Example 2

### Identification and Cloning of a Lipase Gene from Plectosphaerellaalismatis

Chromosomal DNA from Plectosphaerellaalismatis was isolated by QIAamp DNA Blood Mini Kit (Qiagen, Hilden, Germany). 5 ug of chromosomal DNA were sent for sequencing at FASTERIS SA, Switzerland. The genome sequences were analyzed for open reading frames encoding lipolytic enzymes and the Plectosphaerellaalismatis lipase was identified. This Plectosphaerellaalismatis lipase gene was amplified through PCR reaction. For PCR reaction, 20 pmol of primer pair (each of the forward and reverse) were used in a PCR reaction composed of 1 μl of SEQ ID NO: 1 comprising plasmid DNA, 10 μl of 5×GC Buffer, 1.5 μl of DMSO, 2.5 mM each of dATP, dTTP, dGTP, and dCTP, and 0.6 unit of Phusion™ High-Fidelity DNA Polymerase (Finnzymes Oy, Espoo, Finland) in a final volume of 50 μl. The amplification was performed using a Peltier Thermal Cycler (M J Research Inc., South San Francisco, Calif., USA) programmed for denaturing at 98° C. for 1 minutes; 10 cycles of denaturing at 98° C. for 15 seconds, annealing at 65° C. for 30 seconds, with 1° C. decrease per cycle and elongation at 72° C. for 90 seconds; and another 26 cycles each at 98° C. for 15 seconds, 60° C. for 30 seconds and 72° C. for 90 seconds; final extension at 72° C. for 10 minutes. The heat block then went to a 4° C. soak cycle.

The PCR products were isolated by 0.7% agarose gel electrophoresis using TBE buffer where the product band of 1.1 kb was visualized under UV light. The PCR product was then purified from solution by using a GFX PCR DNA and Gel Band Purification Kit (GE Healthcare, Buckinghamshire, UK) according to the manufacturer's instructions.

Plasmid pCaHj505 (WO2013029496) was digested with BamHl and Xhol from NEB (New England Biolabs, Frankfurt am Main Germany) following manufacturer's recommendations, and the resulting fragments were separated by 0.7% agarose gel electrophoresis using TBE buffer, and purified using an GFX PCR DNA and Gel Band Purification Kit (GE Healthcare, Buckinghamshire, UK) according to the manufacturer's instructions.

60 ng of this purified PCR product were cloned into 200 ng of the previously digested expression vector pCaHj505 by ligation with an IN-FUSION™ CF Dry-down Cloning Kit (Clontech Laboratories, Inc., Mountain View, Calif., USA) according to the manufacturer's instructions.

A 2.5 μl volume of the diluted ligation mixture was used to transform E. coli TOP10 chemically competent cells (described in Strains). 4 colonies were selected from LB agar plates containing 100 ug of ampicillin per ml and confirmed by colony PCR with vector primers. The Plectosphaerellaalismatis lipase synthetic sequence was verified by DNA sequencing with vector primers (by SinoGenoMax Company Limited, Beijing, China). The plasmid comprising SEQ ID NO: 1 was selected for protoplast transformation and heterologous expression of its encoded lipase in an Aspergillus oryzae host cell MT3568 (described in the strain chapter). The SEQ ID NO: 1 comprising colony was cultivated overnight in 3 ml of LB medium supplemented with 100 ug of ampicillin per ml. Plasmid DNA was purified using a Qiagen Spin Miniprep kit (Cat. 27106) (QIAGEN GmbH, Hilden, Germany) according to the manufacturer's instructions.

### Example 3

### Transformation of Aspergillus oryzae with the Gene Encoding a Lipase from Plectosphaerellaalismatis and Selection of the Best Transformants

Protoplasts of Aspergillus oryzae MT3568 (see strains chapter) were prepared according to WO95/002043. 100 μl of protoplasts were mixed with 2.5-10 ug of the Aspergillus expression vector comprising SEQ ID NO: 23 and 250 μl of 60% PEG 4000, 10 mM CaCl₂, and 10 mM Tris-HCl pH7.5 and gently mixed. The mixture was incubated at 37° C. for 30 minutes and the protoplasts were spread onto COVE sucrose plates for selection. After incubation for 4-7 days at 37° C. spores of 4 transformants were inoculated into 3 ml of YPM medium. After 3 days cultivation at 30° C., the culture broths were analyzed by SDS-PAGE using Novex® 4-20% Tris-Glycine Gel (Invitrogen Corporation, Carlsbad, Calif., USA) to identify the transformants producing the largest amount of recombinant lipase from Plectosphaerellaalismatis.

The hydrolytic activity of the lipase produced by the Aspergillus transformants was investigated using olive oil/agarose plates (1% protein grade agarose; 1% olive oil; 0.008% brilliant green; 50 mM Hepes; pH7.2). 20 μl aliquots of the culture broth from the different transformants, buffer (negative control), were distributed into punched holes with a diameter of 3 mm and incubated for 1 hour at 37° C. The plates were subsequently examined for the presence or absence of a dark green zone around the holes corresponding to lipolytic activity.

Based on those two selection criteria, spores of the best transformant were spread on COVE-2 μlates for re-isolation in order to isolate single colonies. Then a single colony was spread on a COVE-2 tube until sporulation.

### Example 4

### Fermentation of Aspergillus oryzae Transformed with the Gene Encoding a Lipase from Plectosphaerellaalismatis

Spores from the best transformant were cultivated in 2400 ml of YPM medium in shake flasks during 3 days at a temperature of 30° C. under 80 rpm agitation. Culture broth was harvested by filtration using a 0.2 μm filter device. The filtered fermentation broth was used for enzyme characterization.

### Example 5

### Preparing New Lipases

Additional lipases from following microorganisms were cloned and produced using methods essentially as described in the examples 2-4. In total 5 new lipases were cloned from microorganisms as outlines in table 2 and enzyme prepared for each lipase.

TABLE 2

| | Origin | Time for | |
|---|---|---|---|
| Species | (country) | sampling | Sequence |
| Plectosphaerellaalismatis | Soil, China | 1998 | SEQ ID NO: 1/2 |
| | | | SEQ ID NO: 23/24 |
| Nectria sp. | Yunnan | October | SEQ ID NO: 3/4 |
| | province | 2003 | |
| | China | | |
| Acremonium sp. | Soil, China | 2013 | SEQ ID NO: 5/6 |
| Plectosphaerellaalismatis | Soil, China | 1998 | SEQ ID NO: 9/10 |
| Fusarium solani | Soil China | 1999 | SEQ ID NO: 13/14 |

### Example 6

### Lipase Activity in Wheat Flour Slurry

The lipases described in Examples 2-5 and the lipases listed in table 3 were prepared as described above and tested for lipase activity in wheat flour slurry.

TABLE 3

| Source | Reference | Sequence |
|---|---|---|
| Mucor wutungkiao | WO 2017/093318 | SEQ ID NO: 7/8 |
| Mucor circinelloides | WO 2014/147127 | SEQ ID NO: 11/12 |
| Trichoderma atroviride | WO 2014/081884 | SEQ ID NO: 15/16 |
| Penicillium sp. | WO 2018/099965 | SEQ ID NO: 17/18 |
| Humicolainsolens | WO 2015/085920 | SEQ ID NO: 19/20 |

For determining lipase activity, each lipase was diluted to 100, 50, 25 and 12.5 μg/ml using 0.01% Triton X-100. Then 30 μl of the diluted lipase samples were added to wells of a 96 well microtiter plate containing 150 μl 20% w/w wheat flour slurry preheated to 38° C. resulting in concentrations of 100, 50, 25 and 12.5 μg lipase per g wheat flour. After 20 min incubation at 38° C. with agitation, the reaction was stopped by adding 50 μl stop reagent (1 M phosphoric acid, 7.5% Triton X-100). After heating for 30 min at 50° C. to solubilize the free fatty acids, the microtiter plates were centrifuged for 1 min. Concentrations of free fatty acids in the supernatants were determined using a NEFA kit (Non-Esterified Fatty Acids (NEFA), FUJIFILM Wako Diagnostics Corporation, CA, USA). After 2-fold dilution with 1% Triton X-100, 10 μl diluted supernatant was mixed with 50 μl 0.2 M MES pH 7 and 50 μl R1 reagent from the NEFA kit. After 15 min incubation at room temperature absorbance at 546 nm was read. Then 25 μl R2 reagent from the NEFA kit was added, and after 15 min incubation at room temperature with agitation absorbance at 546 nm was read again. Difference in absorbance at 546 before and after addition of R2 reagent was used in calculation of released concentrations of free fatty acids in combination with results from a standard curve with oleic acid. The commercially available lipoaseLipolase™ was included as control enzyme.

Results are shown in table 4.

TABLE 4
Release of FFA (mM)

| | Dosage | | | |
|---|---|---|---|---|
| | 100 μg/g | 50 μg/g | 25 μg/g | 12.5 μg/g |
| Lipase | wheat | wheat | wheat | wheat |
| SEQ ID NO: 2 | | | | |
| SEQ ID NO: 4 | 0.447 | 0.450 | 0.411 | 0.386 |
| SEQ ID NO: 6 | 0.369 | 0.365 | 0.380 | 0.375 |
| SEQ ID NO: 8 | 0.342 | 0.186 | 0.312 | 0.254 |
| SEQ ID NO: 10 | 0.503 | 0.376 | 0.454 | 0.381 |
| SEQ ID NO: 12 | 0.327 | 0.223 | 0.245 | 0.080 |
| SEQ ID NO: 14 | 0.303 | 0.215 | 0.055 | 0.110 |
| SEQ ID NO: 16 | 0.249 | 0.217 | 0.093 | 0.071 |
| SEQ ID NO: 18 | 0.203 | 0.175 | 0.145 | 0.093 |
| SEQ ID NO: 20 | 0.164 | 0.146 | 0.079 | 0.060 |
| SEQ ID NO: 24 | 0.333 | | 0.289 | 0.319 |
| Lipolase ™ | 0.267 | 0.222 | 0.157 | 0.107 |

The results show that the new lipases have high activity in a wheat slurry, almost equal to or even better that the commercial lipase Lipolase™.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A process for separating wheatflour into two or more fractions including a gluten fraction and a starch fraction, comprising the steps of:
a) mixing wheat flour and water;
b) adding one or more polypeptide(s) having lipase activity;
c) incubating the mixture for a predefined period of time;
d) separating the mixture into two or more fractions including a gluten rich fraction and a starch rich fraction; and
e) recovering the two or more fractions including a gluten rich fraction and a starch rich fraction;
wherein the one or more polypeptide(s) having lipase activity is (are) selected among polypeptides having lipase activity and having a sequence identity to one of SEQ ID NO: 2, 4, 6, 8, 10, 12, 14, 16, 18, 20 or 24 of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

2. The process of claim 1, where in step a) the water and wheat flour is mixed in a ratio of 0.1-3Liter of water per kg wheatflour.

3. The process of claim 1, wherein the one or more polypeptides having lipase activity is added in amounts of 0.1-500 μg enzyme protein per gram wheatflour (μg EP/g wheat).

4. The process according to claim 1, wherein a xylanase is added together with the one or more polypeptides having lipase activity.

5. The process of claim 4, wherein the xylanase is selected from xylanases belonging to the GH8, GH10 or GH11 families.

6. The process of claim 5, wherein the xylanase is a GH8 xylanase and have a sequence identity to SEQ ID NO: 21 of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

7. The process of claim 5, wherein the xylanase is a GH10 xylanase and have a sequence identity to SEQ ID NO: 22 of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

8. The process according to claim 4, wherein the xylanase is added in an amount of 0.0005 to 1.5 mg enzyme protein per g wheatflour.

9. The process according to claim 1, wherein the incubation in step c) is performed for 5 minutes to 8 Hours.

10. The process according to claim 1, wherein step d) is performed in a three-phase separator and provides a gluten rich fraction, a starch rich fraction and a pentosane/fiber rich fraction.

11. The process according to claim 1, having one or more benefits compared to a similar process without addition of a polypeptide having lipase activity selected among: reduced viscosity in the wheatflour slurry, higher protein recovery and higher throughput in the separation step.

12. A polypeptide having lipase activity, selected from the group consisting of:
(i)
(a) a polypeptide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2;
(b) a polypeptide encoded by a polynucleotide that hybridizes under low stringency conditions with the mature polypeptide coding sequence of SEQ ID NO: 1, or the full-length complement thereof;
(c) a polypeptide encoded by a polynucleotide having at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1;
(d) a variant of the mature polypeptide of SEQ ID NO: 2 comprising a substitution, deletion, and/or insertion at one or more positions; and
(e) a fragment of the polypeptide of (a), (b), (c), or (d) that has lipase activity;
(ii)
(a) a polypeptide having at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4;
(b) a polypeptide encoded by a polynucleotide that hybridizes under medium stringency conditions with the mature polypeptide coding sequence of SEQ ID NO: 3, or the full-length complement thereof;
(c) a polypeptide encoded by a polynucleotide at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 3;
(d) a variant of the mature polypeptide of SEQ ID NO: 4 comprising a substitution, deletion, and/or insertion at one or more positions; and
(e) a fragment of the polypeptide of (a), (b), (c), or (d) that has lipase activity;
(iii)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6;
(b) a polypeptide encoded by a polynucleotide that hybridizes under low stringency conditions with the mature polypeptide coding sequence of SEQ ID NO: 5, the cDNA sequence thereof or the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 5 or the cDNA sequence thereof;
(d) a variant of the mature polypeptide of SEQ ID NO: 6 comprising a substitution, deletion, and/or insertion at one or more positions; and
(e) a fragment of the polypeptide of (a), (b), (c), or (d) that has lipase activity;
(iv)
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 10;
(b) a polypeptide encoded by a polynucleotide that hybridizes under low stringency conditions with the mature polypeptide coding sequence of SEQ ID NO: 9, the cDNA sequence thereof or the full-length complement thereof;
(c) a polypeptide encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 9 or the cDNA sequence thereof;
(d) a variant of the mature polypeptide of SEQ ID NO: 10 comprising a substitution, deletion, and/or insertion at one or more positions; and
(e) a fragment of the polypeptide of (a), (b), (c), or (d) that has lipase activity; and
(v)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 14;
(b) a polypeptide encoded by a polynucleotide that hybridizes under low stringency conditions with the mature polypeptide coding sequence of SEQ ID NO: 13, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 13 or the cDNA sequence thereof;
(d) a variant of the mature polypeptide of SEQ ID NO: 14 comprising a substitution, deletion, and/or insertion at one or more positions; and
(e) a fragment of the polypeptide of (a), (b), (c), or (d) that has lipase activity;
(vi)
(a) a polypeptide having at least 90%, at least 91%, at least 92%, at least 93%, at least 94%, at least 95%, at least 96%, at least 97%, at least 98%, at least 99% or 100% sequence identity to the mature polypeptide of SEQ ID NO: 24;
(b) a polypeptide encoded by a polynucleotide that hybridizes under low stringency conditions with the mature polypeptide coding sequence of SEQ ID NO: 23, the cDNA sequence thereof, or the full-length complement thereof;
(c) a polypeptide encoded by a polynucleotide having at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 23 or the cDNA sequence thereof;
(d) a variant of the mature polypeptide of SEQ ID NO: 24 comprising a substitution, deletion, and/or insertion at one or more positions; and
(e) a fragment of the polypeptide of (a), (b), (c), or (d) that has lipase activity.

13. The polypeptide of claim 12, comprising or consisting of SEQ ID NO: 2, 4, 6, 10, 14 or 24; or the mature polypeptide of SEQ ID NO: 2, 4, 6, 10, 14 or 24.

14. The polypeptide of claim 13, wherein the mature polypeptide is amino acids 20 to 413 of SEQ ID NO: 2; amino acids 16 to 339 of SEQ ID NO: 4; amino acids 16 to 339 of SEQ ID NO: 6; amino acids 18 to 343 of SEQ ID NO: 10, amino acids 17 to 185 of SEQ ID NO: 14; or amino acids 20 to 339 of SEQ ID NO: 24.

15. The polypeptide of claim 12, which is a variant of the mature polypeptide of SEQ ID NO: 2, 4, 6, 10, 14 or 24 comprising a substitution, deletion, and/or insertion at one or more positions.

16. The polypeptide of claim 12-14, which is a fragment of SEQ ID NO: 2, 4, 6, 10, 14 or 24, wherein the fragment has lipase activity.

17. A composition comprising the polypeptide of claim 12.

18. A whole broth formulation or cell culture composition comprising the polypeptide of claim 12.

19. A polynucleotide encoding the polypeptide of claim 12.

20. A nucleic acid construct or expression vector comprising the polynucleotide of claim 19 operably linked to one or more control sequences that direct the production of the polypeptide in an expression host.

21. A recombinant host cell comprising the polynucleotide of claim 19 operably linked to one or more control sequences that direct the production of the polypeptide.

22. A method of producing a polypeptide having lipase activity, comprising cultivating the host cell of claim 21 under conditions conducive for production of the polypeptide.

23. The method of claim 22, further comprising recovering the polypeptide.


SEQUENCE LISTING (24 sequences)

SEQ ID NO: 1 - DNA, 1842 bp, Plectosphaerella alismatis
  CDS (101)..(1342)
gtatcctcaccctgaataacgtgcgacgagccatacgatatccacgccctatcctgagatcgacatccttccgtagagcctacaccccgaacccgccgccatggtccgactgttgcaactgggcggtctgctcttcgctgccgccctcgcctcagcggccgaccaacaccctctaaagagaccgcaagatgacacgccgcaacccgtctctgtcgccctcttctcctccctcgaacgcatgtcccgccttgtcgatattgcctactgcgtcggcacgacaggcgtatccgaaccattctcctgcgcctcgcgctgcagggagttcccgaccctgacactcgcgacagtctggaacaccggcatcttgatgagcgacagctgcggctacatcgccgtcgaccacggcacgcgccgccccgacaccgacaaggacgcgctcgaccggctcggcgaaaaggcaatcatcgtggcctttcgcggcacctacagcatcgccaataccgtggtcgatctgagtaccgtgccgcaggaatacgtcccatacccctcacccgacgacggcggtgacgcgcccagggagccgcagcatcgctgcgacaattgcacggttcacagcggcttcctggagtcgtggcagcaggctcgcaagctggtggtgccggagctgcaggcgctcaaggagaaataccccgactaccccgtgcagctcgtcggccacagcctgggcggcgcggtcgccatgctcgccgcgctggagctgaccgtgtcactcgggtggaacgacacgttgacgacaacgtttggcgagcccaaggtcggcaaccagggcctgagcgattacgtcgacgccgtcttccagctctctgagaaggacgacacggaccccgagaagcgcgtgtaccgccgcgtgacgcacctggacgacccggtgccgatgctgccgctgacagaatggggatacagaccgcacggcggtgagttcttcatcaacaagaaagagctgtcgccctcgctggaaaacgtcgtcatctgccgcggtgccggggaccggaactgcgtcgctcagggcgatgtccaccctgaagacctggagcagatccagacgctggccgatgatccagatgcggagctggaaccggagtttgaagttgaaaaggacgacggcgacaagatacgggtgaccaagaggttcccggcccgcttcaatctctggcagctcttcttcgcccaccgagactacttttggaggatcgggctgtgcatccccggcggcgatccggctgactggggccggaagccaaactaccccgtacctggcgacgacttaccggacgagctctagggggatgttcgtgggcctcgaaggcgatcggaactgtagaaaaaagtctgtgtgattaggtagtagcaggagtcgaggcgagactggtgattgggtactgccctggcgttcttgggaaggctctgtaatgagaatgaagacatgatttgaccgttcatacaagggtgtgttgcacggctgtgatgctgatcctcatgtcacgggaggcctcgggggaggagggaggagcagggctgaaggagcaggaggtgtttggcagccacgtatcatggtatgaagataggtacgtagttctctgggaatcagattgttgttcggtatctaaagccaaccttgattatgccacagccacaacggccactacagattcggcggtgtttaggcaccagccatcgacagcttgcttcatcaggccaattcccgcataagcaacagtcagcccccacccggtcaggaggactcgccagcacgagatgacgcgactacgcgtcccctattgc

SEQ ID NO: 2 - protein, 413 aa, Plectosphaerella alismatis
MVRLLQLGGLLFAAALASAADQHPLKRPQDDTPQPVSVALFSSLERMSRLVDIAYCVGTTGVSEPFSCASRCREFPTLTLATVWNTGILMSDSCGYIAVDHGTRRPDTDKDALDRLGEKAIIVAFRGTYSIANTVVDLSTVPQEYVPYPSPDDGGDAPREPQHRCDNCTVHSGFLESWQQARKLVVPELQALKEKYPDYPVQLVGHSLGGAVAMLAALELTVSLGWNDTLTTTFGEPKVGNQGLSDYVDAVFQLSEKDDTDPEKRVYRRVTHLDDPVPMLPLTEWGYRPHGGEFFINKKELSPSLENVVICRGAGDRNCVAQGDVHPEDLEQIQTLADDPDAELEPEFEVEKDDGDKIRVTKRFPARFNLWQLFFAHRDYFWRIGLCIPGGDPADWGRKPNYPVPGDDLPDEL

SEQ ID NO: 3 - DNA, 1159 bp, Nectria sp.
  CDS (1)..(1020)
atgcgtgtcctccctttcctctccgtcgtcggcgtggcttcagccgcctccatcaagagctatcttcgtgctcttgaggatcgagctgtcactgtgacttctacgaaccttgccaacttcaagttctatgcccagcacgccgctgctgcgtactgcaactgggatgccacagccggggctgccatttcatgctcggggtcctgcgcaaccgtcgagagcaacggtgccaaagtcgtagcatcttttggaggcgaggatactggcattgggggctacgtctcgacagacgcaacccgcaaggaaatcgtcatctcagtgcgcggcagcagcaacgtccgcaactggatcaccaacctcgagttcctgttcagttcatgcagcgacctctcctccagctgcaaggcccactcgggcttcaaggaggcttgggacgaggtgtccacggccgcaaaggctgccatcgccacggctaagacggccaatcccacctacaccgtcgtcgctaccggccactccctgggcggtgccgtcgcgaccctggctgctgcctacctccgcaaggccgggtactcggtcgacctgtacacgtttggctcgccgcgcgtgggcaacgactacttcgccaactttgtcaccagccaaaccggcgccgaataccgcctcacgcacctcgacgacccagtccctcgtctgccgcccatcatctttggttaccgccacacgtctcccgagtactggctctctactggagactcggacacgacggcgtacggcatcgccgacatcaaggtgtgtgaaggcattgccaacattggctgtaacgcgggcaccattggccttgacattgaggcgcacctcatctatttccaagatctctctggttgcacgggcactttcacctggaagcgcgacgacctgaccgacgccgagctcgaggagaaggtgaacaactgggccctccaggatgtggaatacatctccaacaagacttcggccaggcgatggaaatcggcccagtgatgaagcccggcaggttcaagtgaaattttgttctgtttataattggaagcatggaaatcaatgtatgatagcgtacatacgtcaaataagtccagagagttggataatctgcgagaaaaaaaaaaaaaaaaaaaaaaaa

SEQ ID NO: 4 - protein, 339 aa, Nectria sp.
MRVLPFLSVVGVASAASIKSYLRALEDRAVTVTSTNLANFKFYAQHAAAAYCNWDATAGAAISCSGSCATVESNGAKVVASFGGEDTGIGGYVSTDATRKEIVISVRGSSNVRNWITNLEFLFSSCSDLSSSCKAHSGFKEAWDEVSTAAKAAIATAKTANPTYTVVATGHSLGGAVATLAAAYLRKAGYSVDLYTFGSPRVGNDYFANFVTSQTGAEYRLTHLDDPVPRLPPIIFGYRHTSPEYWLSTGDSDTTAYGIADIKVCEGIANIGCNAGTIGLDIEAHLIYFQDLSGCTGTFTWKRDDLTDAELEEKVNNWALQDVEYISNKTSARRWKSAQ

SEQ ID NO: 5 - DNA, 2131 bp, Acremonium sp.
  CDS (501)..(594)
  CDS (648)..(813)
  CDS (872)..(1628)
gaatctcgcgatcggttgaccatgtggaaaccctgtttcccgccttttttttcttgatcgtgctctttttttttttccctttcgcttttggctcgcacaccggcttttgttcccccgcaaaagctcggacgttgcgcccttctgagaaaggtgcgcgtctgggctggggtgggtttcagtgcataggttgacagaacttccccatggggaccggggatgtcgtgagaaatgggtgtaacaggaccatttgtaagtgtcgcggacggaagtaacggatagctgagtagaatgcttgcatacagctcccgtgttggtgtgatataagtatctgcgtcaattcctaagatatgtctcgcgggtgtctttgagtcgttgtccagatccctgtctcctccaagtgcctgatctcggtcccccttctcccttgcggctctcctcactacttgaggtatacccatcttgaggtacgcagcccaagttgatccaacagaaacagcaacatgagaggacttttcttcttgtcgctggcctctctggccatcgcatctccccttggttctgttgagaagtactcaagagctcttaagagcaagtgtaggttaaaattccactgctttcttgagtctgatagactgacagtttgatagctgttgctgttacggagcagactcttgataattttcaatattatgtccagcactcagcggccgcgtattgcaatgtcaggccgagctctggtgctcgcattacgtgcggtaatgatgtctgtcctgaccttgaaggcaatgccgtcacagccgtgagcggcttcaggtaagtgatcttcaaggcatgattggaaatgaactcgtgctaacaacatgccacccagcggtctcatcactggcattgccggttatgttgccacagaccacgcccggaaagagattgtcctctcggtgcgcggcagcaacaacgtccgtaacttcatcacggatgttgtctttgcctggacctcctgcccctttgtttccgactgcaaagtccacaagggtttccaagccgcttgggatgagatctccagtgcagcccagagcgccatcaagtccgcccgcgccgccaacccgggctaccgtgtcgtagccacaggccactcccttggtggcgcagttgccaccctcggtgccgtttacatgagacggaatggtattacggtcgatgtgtactcatacggcagccctcgtgtcggtaacgacaagtttgccaactttgtctcaaaccagggcgcaggtgagttccgcgtgacacacacggatgaccctgtcccgaggttgcctcctattatcttcgggtatcggcacacgacgccggagtactggctttcgaccgatgccagctccaatgagtaccctctcaacgatattcgcgtttgtgaaggtattgccaacatcaggtgcaatggtggaacgtttggcctcaatgttccgagtcaccttcagtatttcatcgaggtgtcgggatgctcgcctatcaatatcaagaggctggcagtatcttcacaatctgcaagcgatgatatctccgacgaggatctagaggagaggttgaacagctggagccagatggatcaggactatgtcaatcacgcgtaaatggcaagaggctggggacttggatcctaaagacaaagcgtatgaatgatttatgatcttccttgaactccatggcaaactctgaaaggatatatgaaccgattctaaactttgtgcgccttcttcgccccggatgcgtcaaattgtacattacaataacttacgatgtctaagcatcgccttccgtgaccatgccttgagcgtttggtaccccgatgagcactgcacctctggggactcggacctgcctaaccttcatgcaaccgaacacgttgaccttcttaaccttgggacaggatcggaagatggagcccagaacgaagtcgttcacgttctcacaaaagctgatctccaagtccaccaactcgggataggtagcatcttcggagaacgcttcctcaaaggcctgctgagaaatgtggcggcatccgtgaatgttcagaaccttcattcttgatccagaatgttccatgagggctttgaaaccctcgtcgcaaagg

SEQ ID NO: 6 - protein, 339 aa, Acremonium sp.
MRGLFFLSLASLAIASPLGSVEKYSRALKSKSVAVTEQTLDNFQYYVQHSAAAYCNVRPSSGARITCGNDVCPDLEGNAVTAVSGFSGLITGIAGYVATDHARKEIVLSVRGSNNVRNFITDVVFAWTSCPFVSDCKVHKGFQAAWDEISSAAQSAIKSARAANPGYRVVATGHSLGGAVATLGAVYMRRNGITVDVYSYGSPRVGNDKFANFVSNQGAGEFRVTHTDDPVPRLPPIIFGYRHTTPEYWLSTDASSNEYPLNDIRVCEGIANIRCNGGTFGLNVPSHLQYFIEVSGCSPINIKRLAVSSQSASDDISDEDLEERLNSWSQMDQDYVNHA

SEQ ID NO: 7 - DNA, 1134 bp, Mucor wutungkiao
  CDS (1)..(1134)
atggtctcgttcgtctccatttcccagggcatctcgttgttcatcattgtctcctcgatgttgacgggttcgacgaacgcagcgcctacatcgaccaacaagacaacggaagccactactttcactctccctcccctcatctcgtcgcgcatcattgcacccaaagtgccgtccggctcccacgatgtggaagcagcgaacatcctcaagaacaaagaatggttcgaagcaaacggaggaaaactcgccgtgactaagcgggatgacgagaccgtcggaggcatgtacatggatctcccgtccaactcccctgcaattcctgccgtctccaacgagtcgaccgtggtgatcgccactacggcacagatcactgagttgaagatgtatgcaggcatcgcatccacagcctattgtcgatcggtcgtgcccctcaacgcctggacctgtacgaactgtttgaagttcgtgccggatggcaaactcatcacaaccttctcgtccttgattaccgataccaacggattcgtcctcaggtcggatgcccagaagactatctatctcgtgttccgaggaaccaactcgattcggtcggcgattacggatctcatcttcgacttcaccgactatactcccgtctccggtgccaaggtccacaaaggtttctacgcatcgtataaggaggtggtgaactcgtacttcccctacattcagtcgcagttgaccgcctaccccacctataaggtgatcgtgacaggccactccctcggaggagcccaggccctcttggcaggcatggacctctaccagcgggaatcccgactctccaagtccaacttggcaatctatacagtcggttgtcctcggacaggtaaccctgcgttcgcatactacgtcgagtccacgggaatcacgttctccaggtcggtcaacaaccgggatatcgtcccccatgtccctccgcaggccttcggttacctccatcccggagtggaggcgtgggcacgctcctcgtccaaggtccagatttgtactcccgacatcgaaacctccttgtgttccaactcgatcgtgccgttcacgtcgttcaccgatcacttgacttactatgacatcaacgagggcctctgtctctaa

SEQ ID NO: 8 - protein, 377 aa, Mucor wutungkiao
MVSFVSISQGISLFIIVSSMLTGSTNAAPTSTNKTTEATTFTLPPLISSRIIAPKVPSGSHDVEAANILKNKEWFEANGGKLAVTKRDDETVGGMYMDLPSNSPAIPAVSNESTVVIATTAQITELKMYAGIASTAYCRSVVPLNAWTCTNCLKFVPDGKLITTFSSLITDTNGFVLRSDAQKTIYLVFRGTNSIRSAITDLIFDFTDYTPVSGAKVHKGFYASYKEVVNSYFPYIQSQLTAYPTYKVIVTGHSLGGAQALLAGMDLYQRESRLSKSNLAIYTVGCPRTGNPAFAYYVESTGITFSRSVNNRDIVPHVPPQAFGYLHPGVEAWARSSSKVQICTPDIETSLCSNSIVPFTSFTDHLTYYDINEGLCL

SEQ ID NO: 9 - DNA, 1337 bp, Plectosphaerella alismatis
  CDS (101)..(194)
  CDS (246)..(411)
  CDS (466)..(1234)
atctcacccatcaagggcttctcatctggttgtctctcgctcctcaacccgactcactgccactttgctaccagtctctcgccggacctagtcgctcacaatgatcgccaacaactggatccttagccttttggctgtcactgccgctgcttcgccactccagcaggcgcccgagtttgcccttgaagcaaggcgtgcgtgagctctggcttcaacacagctgcccctagctaaattcctcccagaaagcagcgtcaccacccagcagctggccgacttcaagatctacgccgaatatgccgccgcctcctactgcaacggcaaaaacacccccggccagctcatcgcctgctctggcagtgcctgcaaccaggtctcggctaaccgtcccgtcaccgtggcttccttctcgtgagcactcccgctccatcatgaacttctacttcgtgactgacccgaccccagcggcttcgtgactggtattgaaggctttgttgccacagacccggtgcgccgagagatcatcctctccattcgcggaagctccaacgtccgcaattggatcaccaactttcagttcctgtccggcgcctgtgacctcgtcaggcgatgcagggtccactcgggcttcaacaacgcctggagcgagatctcgaccaacgcacaaaacgccgtcagacaggcccgctccgcgaaccccgggtaccgcgtcgtcacaaccggtcactcactcggtggcgccgtggcgactctcgctgctgcccatctccgcgcgggaggcattcccaccgagctctacacctacggcgcacctcgtgttggcaatgaagagttcgccaactttgtcagccgccagccgggcggtcactaccgcatcacccacggcgccgaccccgttcccaaggtgccccctttcgcctttgggtacaggcacaccacccccgagtactggctcaacggtggttcgtcacgcaccatcgactacggcatcagggacatcaaggtgtgcaccggcactctgagctttggctgcaacaacgggctcgatattccggatgtcgtctcgcacatgttctacgtcaacagcatgggaggctgcagccctggtggattcgacattaggcgcatgggcgatgaggcggacgagatcgatgaggcattcagccggatgattgaggtcgatcgtccgtacattcaggagaacaggatgcctagcaacaacagcatgacggtctagggtggaggtagagcaatccagatcagaaagttttggatggcaagacagatggacacgtgtccaggctgaagccggatattggagaaatgcggtcctaggg

SEQ ID NO: 10 - protein, 343 aa, Plectosphaerella alismatis
MIANNWILSLLAVTAAASPLQQAPEFALEARQSSVTTQQLADFKIYAEYAAASYCNGKNTPGQLIACSGSACNQVSANRPVTVASFSGFVTGIEGFVATDPVRREIILSIRGSSNVRNWITNFQFLSGACDLVRRCRVHSGFNNAWSEISTNAQNAVRQARSANPGYRVVTTGHSLGGAVATLAAAHLRAGGIPTELYTYGAPRVGNEEFANFVSRQPGGHYRITHGADPVPKVPPFAFGYRHTTPEYWLNGGSSRTIDYGIRDIKVCTGTLSFGCNNGLDIPDVVSHMFYVNSMGGCSPGGFDIRRMGDEADEIDEAFSRMIEVDRPYIQENRMPSNNSMTV

SEQ ID NO: 11 - DNA, 1752 bp, Mucor circinelloides
  CDS (101)..(754)
  CDS (810)..(867)
  CDS (923)..(1019)
  CDS (1073)..(1257)
  CDS (1319)..(1399)
  CDS (1463)..(1536)
  CDS (1614)..(1649)
gcctattatttgtatataaataccacattgcaaagacccatcgacagtttctcttcatcagcttcctcaagccttctccccttcctttccttctgtcaacatggtcttgatctcctctcttaccaaagacgtcaagtttatccttgccgcctcttacgtcctctttgctgctgtctctgatgccgctcctaccgccatccctctcattgaaagatccactgcttcttcaggcaatgttactcacgtcaatggcaccttgacggatgccgatgtcactcttcctcccttgatccccaagcgtgtcattcctgctaccaagattcctcatgattacgatgttgaaaccgagtatatcaccaagaacacggaatggtacaatcaacatggtggcaactacacccttaccaagcgtgatgaaatcgagacagtcggtaactttaccatggatcttcctcccaatcctcctcctcttcccgcttatcccaaggatgtcttggttgtcaaggctgatgccaacaagatcaaggagcttaccaagtatgctggtattgctgctgctgcttactgccgtgatgttgtacctgccaacaactggaagtgcaagcaatgtttgaaacaggtaccagatggcaagcttatcaagacttttacctcttttgtatctgataccaatggttttgtcttgagaagtgacaaggaaaaggtcatctaccttgtcttccgtggcaccaattctatcagaagcagcattgctgtaagtaatgagcataacaactacgcaaatttgggctaaattcgatttgcaataggatatccaatttgattttaccaattaccctaatgtcaagggtgccaaggttcatagaggtgcgttcatttccaagcgttatagtctatgagactaatatcctttccctgctaggtttcttgaactcttataatgaagtcgtccgctccttcttccctgatatccaagaccaaatcactgcctttccttcttacaaagttatcgttaccgggtaagtgcaagttaatataccatcatattcacttcatgctcatatttcaatagacattctcttggtggtgctcaagcattgcttgctggcatggacctttaccaaagaatctccacattatctgccaagaatttagccatctacactgttggcatgcccagaactggaaatgccgcgtttgcttattatgttgattcaactggcatccccttatctcgctccgttaatgaaagagacagtgagttggtggttttgagtagagtagatctttggaagctgacgcattattcctcttctagttgtacctcatgttcctcctcaagcttttggctatcttcaccctggcgttgaagcttggaccagatcttctggaaatgttcgtaagtattgatgatagcacgtgtcatgattatctattcacgcgttatatacgtatcatgtagaaatctgtaccgagagcattgaatccgacttgtgttccaataccattgtacccttcactaccatcttggatcatttgaggtaagaatcagttgatctttgatatatatttatgttggcctgcaaaattaacgtgcacatattatcattcattttagctattacgacatcaatgaaggtctctgcctctaatctaacagcatatgtaatcaattagataatcagcataaactttcaatgtactcattgggcgtaacatatgtatttgtaacactagtattgatttactgta

SEQ ID NO: 12 - protein, 395 aa, Mucor circinelloides
MVLISSLTKDVKFILAASYVLFAAVSDAAPTAIPLIERSTASSGNVTHVNGTLTDADVTLPPLIPKRVIPATKIPHDYDVETEYITKNTEWYNQHGGNYTLTKRDEIETVGNFTMDLPPNPPPLPAYPKDVLVVKADANKIKELTKYAGIAAAAYCRDVVPANNWKCKQCLKQVPDGKLIKTFTSFVSDTNGFVLRSDKEKVIYLVFRGTNSIRSSIADIQFDFTNYPNVKGAKVHRGFLNSYNEVVRSFFPDIQDQITAFPSYKVIVTGHSLGGAQALLAGMDLYQRISTLSAKNLAIYTVGMPRTGNAAFAYYVDSTGIPLSRSVNERDIVPHVPPQAFGYLHPGVEAWTRSSGNVQICTESIESDLCSNTIVPFTTILDHFSYYDINEGLCL

SEQ ID NO: 13 - DNA, 711 bp, Fusarium solani
  CDS (42)..(382)
  CDS (442)..(655)
ttccttcctcaatcctctatatacacaactggggatccaccatgaagttcagcctttgcctcgtctctctcctgccagtggcatttgccctgccagctggcacaaaggacgagtccgtctctaagcgtcaaagtgccaacacggtgacggatgagctgctcttcagtgtctcgttgcccaccttcactgcgcgacgtaacgcgcgagaccccccaaccctcatctgggactctgatggttgcacctcatctcccgacaatccgtttggcttcccctttgtcccagcctgcaaccgtcacgactttggctaccacaactatcgtgctcagagccgattcactgtgagcgggaaggctcgcattgatagcaacttcaagaccgagtgagttcctcacaaattacttgaatccttttaaataattactgacatgccatctcaagcctgtactatcagtgcgagtcttccagcgtctcgggtgtctgcagggctctggccgacgtctactatgccgccgtccgtgcctttggcggtgatgatgccactcccggcaagaggagtgacagcgagttggtgaaggagtatgaggagaaggtggctatctacaacaagcttgttgaggaagctcaggagaagggagacctccctcgactggactagttgacaaaagtctagcatctcgagatctagagggtgactgacacctggcggta

SEQ ID NO: 14 - protein, 185 aa, Fusarium solani
MKFSLCLVSLLPVAFALPAGTKDESVSKRQSANTVTDELLFSVSLPTFTARRNARDPPTLIWDSDGCTSSPDNPFGFPFVPACNRHDFGYHNYRAQSRFTVSGKARIDSNFKTDLYYQCESSSVSGVCRALADVYYAAVRAFGGDDATPGKRSDSELVKEYEEKVAIYNKLVEEAQEKGDLPRLD

SEQ ID NO: 15 - DNA, 900 bp, Trichoderma atroviride
  CDS (48)..(472)
  CDS (548)..(863)
cttctcttccttcctcaatcctctatatacacaactggggatccaccatgcgatccgcagccattttcacagctctcctggcgggccaggcctttgcttacccgaagccggtcatccagtccaccagcaagcgggattggccttcaatcaatgccttcctcaccgagattgcgaagattatgcccattggtgataccgtcacggctgcttgtgatctgattggagatggtgaagacattgctgccgatctctttggcatctctcaaactgagaatgaggcgtgcggcgacgtaaccgtcttgtttgctcgcggtacttgcgaccctggcaatgtcggagtacttgtcgggcctttcttcttcgagtcgctgcaaaccgcactcggtagcacatctcttggtgttcaaggcttcaactaccctgcgagtgttcagggtttcctgtctggagcggtccagccaggcattgacttgtaagtcacatactcattattcgcttttccactgcaaaaaaccaccagactaaccagctatttcaactcaaatagggccactcaaatcacatctgtcacacagagctgcccgaataccaagcttgtggttggcggctactcccaaggcagcttggtcgtccacaacgcagtgggccacttggatgcagcaaccgcctccaaaatcagcgccgtggtgctctttggcgacccaagagacggacaggctcttcccaacattgctgcttccaaggttctgaccgtctgccacgatggagacaacatctgcgctggtggagacatcatcctgctcccacacttgacatatgccgaggacgcagataccgctgctgcctttgttgcatctcttgtttgagaaatcactcgagatctagagggtgactgacacc

SEQ ID NO: 16 - protein, 247 aa, Trichoderma atroviride
MRSAAIFTALLAGQAFAYPKPVIQSTSKRDWPSINAFLTEIAKIMPIGDTVTAACDLIGDGEDIAADLFGISQTENEACGDVTVLFARGTCDPGNVGVLVGPFFFESLQTALGSTSLGVQGFNYPASVQGFLSGAVQPGIDLATQITSVTQSCPNTKLVVGGYSQGSLVVHNAVGHLDAATASKISAVVLFGDPRDGQALPNIAASKVLTVCHDGDNICAGGDIILLPHLTYAEDADTAAAFVASLV

SEQ ID NO: 17 - DNA, 1544 bp, Penicillium sp.
  CDS (101)..(406)
  CDS (465)..(610)
  CDS (671)..(828)
  CDS (887)..(1041)
ctcgatgccccaggatgagaacggggtcattctttctcaacaggcacgcgaacgcaccagagtcactatctctgcagcatcacattccagaatcagaatcatgatcctccagcttcgatatttggccttgatctttttcgggctgcatgcctatgctgtcccgcttgcggaccgtgaggtgcaccatctcaaggaacgcggcgctgggttgaattcatttctcaattttctcctgagctatctgccagctatcaacacatcaatcacagacgcaactggtctcatcaccgactttgacaagctgctcgggggcctgactggagctcaaacgacgtacaatgagctaggaggtgcctgtacagcatataccgtgatatttgcgcgcggcacggcagagccaggcaatgtaggagatccaaggtcatttttctgtcttcgcctcattactcatgtcgatgctttaggtcggggttctggtcggcccacccttgtttgatgccttggacgacaagtttggatcatcggcacttaccattcagggcgtcaatggctactcggcctctgtccaaggctacttagctggaggggatccaagtggaagcgcgtccatgtgagtaccacaatcaagcgtcctttttcgataatgacagcgccctgattcaggatatagggcaaaccaaattaaagcagcaaaggcccaatgtcccaagacgaagttaatcgcgtcaggctactcccagggctgtcaaattgtgcacaatgcgatctcccagcttgatgctacaaccgcgagctggatttccagcgtactgctgtttggtgacccacgtacgatgagcatccatctattccactaccacgtccgcaattactgaccaaatccaagtcaaaggacaggccctgaagaatgtcccagcctcaagagttttcaccgcatgccacgccctcgatgacatttgcaaggatggcctcattattggaccttcgcatctgacctacgccatcgacgttactaacgcagccaattttgcagcggccgtttagtcaacacgatgcagggcctggtgtccaaacatatcccagttggctggagtttcaattgagccagaccatactcatgataaaggaatgcctccgctatagcgtctgtcctggagacagtgaaatgccagcatggcttctttttgatattccattgcctttgtatctagttcgacatcgagatcttgaaccagtaaatgaaacgtgggcttgaaaagggcgaaaatccatcgtttccccggcagcggtctcttatatcccactcgtctgggctaggtccaggtgtccttaggtaacggagggaaattcttattccatcggcagccacgactattgaaagcaaaagatatgtatactttcagagcagtttacagatggcgcagcccctcaatcgcatctgtagctcagaaatggaccaagtacagcaaaaataaatatggactaagattctacctgcagtattggatgcatcgcactacctccaagatgagaattctgcccaa

SEQ ID NO: 18 - protein, 255 aa, Penicillium sp.
MILQLRYLALIFFGLHAYAVPLADREVHHLKERGAGLNSFLNFLLSYLPAINTSITDATGLITDFDKLLGGLTGAQTTYNELGGACTAYTVIFARGTAEPGNVGVLVGPPLFDALDDKFGSSALTIQGVNGYSASVQGYLAGGDPSGSASMANQIKAAKAQCPKTKLIASGYSQGCQIVHNAISQLDATTASWISSVLLFGDPLKGQALKNVPASRVFTACHALDDICKDGLIIGPSHLTYAIDVTNAANFAAAV

SEQ ID NO: 19 - DNA, 722 bp, Humicola insolens
  CDS (20)..(706)
acacaactggggatccaccatgaagttcttcaccacgatcctctcgactgcctcgttggtcgcagccttgcctgcggcagtggattccaaccacacaccggcagcacccgagctcgtggcacgccagctcggagccatctgcaacgacttggaatcgggttcgcctgacgcgtgtcccgatgcaattctcattttcgcacgaggatcgatggaacccggtaacatgggtatcactgtcggacctgcgttgatcaacggtttgaaggagcatatccccaacatctggtgccagggagtgggtggcccttacgacgcagcgctcgcaaccaacttcttgcctcgcggaacgtcgcaggccaacatcgacgagggaaaaaggctcttccacctcgcccatcagaagtgtcccaacacaccggtggtggcaggaggatactcccagggtgcagcgttgattgccgcagccgtctcggaattgtcgggagcagtgaaggagcaggtcaagggagtcgtcttgttcggatacacccagaacctccagaaccgaggaggcattcccaactatcctcgcgagcgcacgaaggtgttctgtaacgtgggtgatctcgtgtgtacaggcatcccgatcatcactcctgcccacctctcgtataccatccaggcgcccgaggcagcacggttcctcgtcgaccgaattagggcgtagctcgagatctagaggg

SEQ ID NO: 20 - protein, 228 aa, Humicola insolens
MKFFTTILSTASLVAALPAAVDSNHTPAAPELVARQLGAICNDLESGSPDACPDAILIFARGSMEPGNMGITVGPALINGLKEHIPNIWCQGVGGPYDAALATNFLPRGTSQANIDEGKRLFHLAHQKCPNTPVVAGGYSQGAALIAAAVSELSGAVKEQVKGVVLFGYTQNLQNRGGIPNYPRERTKVFCNVGDLVCTGIPIITPAHLSYTIQAPEAARFLVDRIRA

SEQ ID NO: 21 - protein, 407 aa, Basillus sp.
AVHSKTPDILGTTGKNNLNQAYKKYFDTKGDGKGGSLFHYMKDGSAYIASTTDDNLLGNGYYSVKTEGMSYGMMITLQMNDEYKFQKLWDFVRKYMRHSDRNDSLYGYHSWHMKTNGSDVQTIDQNVASDGEVWFAAALMMASGRWGDKKYPYDYKARAQDMLDALAGDGEYANTGKESRVFIKNSKDQRYAMVRFGPYVNWTDPSYHVPAFFELFAKSAKSSQQYFWKDAANKSRTYLSETTFKSVLNNGSTVTNAATGLFPDEAGFDGVSDAAHSSTETDRNFSYDAWRTVSHIAMDHTLWSSADNAYRASEQKAVNKFLTFMKRENYGRTAHEYTLNGTAVKKGSPVGLIAANAGGATAASDASLRTGFANAFNSTYIPEDYYGSCLYMLNSLVANGKFAMYLP

SEQ ID NO: 22 - protein, 384 aa, Aspergillus aculeatus
VGLDQAAVAKGLQYFGTATDNPELTDIPYVTQLNNTADFGQITPGNSMKWDATEPSQGTFTFTKGDVIADLAEGNGQYLRCHTLVWYNQLPSWVTSGTWTNATLTAALKNHITNVVSHYKGKCLHWDVVNEALNDDGTYRTNIFYTTIGEAYIPIAFAAAAAADPDAKLFYNDYNLEYGGAKAASARAIVQLVKNAGAKIDGVGLQAHFSVGTVPSTSSLVSVLQSFTALGVEVAYTEADVRILLPTTATTLAQQSSDFQALVQSCVQTTGCVGFTIWDWTDKYSWVPSTFSGYGAALPWDENLVKKPAYNGLLAGMGVTVTTTTTTTTATATGKTTTTTTGATSTGTTAAHWGQCGGLNWSGPTACATGYTCTYVNDYYSQCL

SEQ ID NO: 23 - DNA, 1077 bp, Plectosphaerella alismatis
atgttgttcaagctccccttcctctccctgctgtccctggccgcggccattcccgtcacagacttcagcagggccctcagcaacatcgacgccaggggtaagcctctctgcctcccgtcccccttgcgtgataacctcgactaacacctctcagacatctccgtctcgcaggccgacctggacaacttccgcttctacgtccaatatgccggtgctgcctactgcaactcggccaacgcccccggcgccctcgtcacctgcgctgcttcggcctgcaagaatgtcgaggccaacggcgctgtgaccgtcgcctcggtcgagggcctgcgttccggcctcggcgccttcgtcgccgtcgaccacacccgccgcgttattgttctggctatccgcggctcggccaacgtgcgcaactggatcaccaacctccagttcaccttcaccagctgcggtgaccttgccaagaactgcaaggttcacaacggcttcggcgatgcctgggccgagatcagcggcactgttctcgacgccatccgcgctacccgcgccgcgaacccgacctatgccgtcgtcgctaccggccacagcctcggcggtgccgtagccaccatcggcgctgcgtacatccgccgcgccgagggcgtccctgttgacatctactcctttggctccccgcgcgccggcaacgactactttgccaacttcgtcaccgctcaggtcggcgccgagttccgtctgacccacggcgccgaccccgtctcccgcctgccacctattctctttggctaccgccacactagccccgagtactggctcaacggcggctcggctaccaccaccgactacgccctcaacaacgttcgtgtctgcgagggcatcgccaacattgggtgcaacggtggcactcttggccttgacatccctgcccacggatactacctccaggacgtctccgcctgcaaccccaagaacgcgcccaccgcccgccagaccgaggtgtccgacgaggagctcgagcagaggctcaacgcctgggtccaccaggacgtcgagtacgtgcaggagcactcggagtag

SEQ ID NO: 24 - protein, 339 aa, Plectosphaerella alismatis
MLFKLPFLSLLSLAAAIPVTDFSRALSNIDARDISVSQADLDNFRFYVQYAGAAYCNSANAPGALVTCAASACKNVEANGAVTVASVEGLRSGLGAFVAVDHTRRVIVLAIRGSANVRNWITNLQFTFTSCGDLAKNCKVHNGFGDAWAEISGTVLDAIRATRAANPTYAVVATGHSLGGAVATIGAAYIRRAEGVPVDIYSFGSPRAGNDYFANFVTAQVGAEFRLTHGADPVSRLPPILFGYRHTSPEYWLNGGSATTTDYALNNVRVCEGIANIGCNGGTLGLDIPAHGYYLQDVSACNPKNAPTARQTEVSDEELEQRLNAWVHQDVEYVQEHSE